(19) United States
(12) Patent Application Publication
(10) Pub. No.: US 2005/0130919 A1
(43) Pub. Date: Jun. 16, 2005

(54) Regulatable promoters for synthesis of small hairpin RNA

(75) Inventors: Zuoshang Xu, North Grafton, MA (US); Xugang Xia, Westborough, MA (US)

Correspondence Address: LAHIVE & COCKFIELD, LLP., 28 STATE STREET, BOSTON, MA, 02109, US

(73) Assignee: UNIVERSITY OF MASSACHUSETTS, Worcester, MA (US)

(21) Appl. No.: 10/894,949

(22) Filed: Jul. 19, 2004

Related U.S. Application Data
(60) Provisional application No. 60/488,510, filed on Jul. 18, 2003.

(51) Int. Cl. A01K 67/027; A61K 48/00; C07H 21/02; C07K 14/47; C12N 15/85

(52) U.S. Cl. 514/44; 435/69.1; 435/455; 435/320.1; 435/325; 530/350; 536/23.1; 800/21

(57) ABSTRACT

The present invention provides compositions for RNA interference and methods of use thereof. The present invention is based on the development of promoters that can be used to regulate shRNA expression spatially (in specific cells) and temporally (at specific times) in cells or transgenic animals that express a recombinase. The compositions and methods of the present invention feature regulatable promoters that allow for inhibition of the expression of target alleles in a spatially and temporally regulatable manner. Thus, the compositions of the present invention are useful for investigating gene functions, both physiologic and pathologic, in specific cell groups and in specific ages, in normal and disease pathways. Functional and genomic and proteomic methods are featured. Therapeutic methods are also featured.

### RELATED APPLICATIONS

This patent application claims the benefit of U.S. Provisional Patent Application Ser. No. 60/488,510, entitled “Regulatable Promoters for Synthesis of Small Hairpin RNA”, filed Jul. 18, 2003. The entire contents of the above-referenced provisional patent application are incorporated herein by this reference.

### GOVERNMENT FUNDING

This invention was made with a Government grant from the National Institutes of Health (R21 Grant No. S111500596A0000). The U.S. Government has certain rights in this invention.

### BACKGROUND OF THE INVENTION

RNAi can mediate sequence-selective suppression of gene expression in a wide variety of eukaryotes by introducing short RNA duplexes (called small interfering RNAs or siRNAs) with sequence homologies to the target gene (Caplen et al., 2001; Elbashir et al., 2001c). Recent experiments indicate that small hairpin RNAs (shRNAs) transcribed in vivo can trigger degradation of corresponding mRNAs similar to the siRNAs (Shi, 2003). These developments raise the possibility that siRNA duplexes or vectors expressing shRNAs (small hairpin RNAs) may be used to block the expression of a toxic gene.

shRNAs can be synthesized from plasmid constructs directly in cells. A common approach uses type III RNA polymerase (Pol III) promoters, one of which is the U6 promoter, which offers several advantages. First, this class of RNA polymerases naturally produces small, non-coding transcripts such as U6 small nuclear RNA (snRNA). Second, their natural transcripts are neither capped at the 5′ nor polyadenylated at their 3′ ends, and therefore resemble siRNA. Third, all of their promoter elements, which include a distal sequence element (DSE), proximal sequence element (PSE) and TATA box, are located 5′ to the transcription initiation site, thereby allowing convenient design of transcript sequences. Fourth, transcription directed by these promoters initiates at defined nucleotides, e.g., a G for the U6 promoter, and terminates when the transcription encounters four or more Ts in succession. Incidentally, the transcripts also carry 3′ overhangs of one to four Us (the termination sequence), a structural feature similar to what has been defined in vitro for effective siRNAs.

The U6 promoter is a strong constitutive promoter. Being able to regulate the U6 promoter (for example, in shRNA expressing constructs) would significantly advance the RNAi field.

Recent work has suggested that Pol II, rather than Pol III, is responsible for synthesis of micro RNAs (miRNA) in vivo. miRNAs are endogenous small RNAs (21-25 nt) that interact with the RISC complex. miRNA are synthesized as large pri-miRNAs, which are processed by endonuclease Drosha in the nucleus to pre-miRNA. The pre-miRNA has a hairpin structure and is exported to the cytoplasm by exportin 5 and Ran GTPase. The pre-miRNA is further processed in the cytoplasm by Dicer to generate a double stranded miRNA, which unwinds to become single stranded RNA and complexes with the RISC to carry out its functions, such as translational repression and RNAi.

This new understanding in the endogenous miRNA mechanism suggests that Pol II promoters may be useful for synthesis of shRNA because they mimics the endogenous miRNA production mechanism. Indeed, previous work has shown that the CMV promoter, a viral Pol II promoter, could be used to synthesize small hairpin RNA in mammalian cells (Zeng, 2003). CMV promoter is a strong but constitutive promoter that is not regulated. It is also a viral promoter that works well in cultured cells but poorly in vivo, particularly in some adult somatic cells. There is, therefore, a clear need in the art for the identification of alternative Pol II promoters which may be useful for shRNA synthesis in vivo.

### SUMMARY OF THE INVENTION

The present invention is based on the development of constructs that can be used to regulate shRNA expression spatially (in specific cells or tissues) and temporally (at specific times) in cells or animals (e.g., transgenic animals). In particular, the invention features constructs that include promoters (e.g., Pol III promoters and Pol II promoters) that are regulatable by a recombinase (e.g., a recombinase co-expressed in a cell or animal expressing a construct of the invention) or by tetracycline or tetracycline analog. Preferred constructs of the present invention, recombinase or tetracycline (e.g., tetracycline analog) regulatable promoters, provide for inhibition of the expression of mutant target alleles in a spatially and temporally regulatable manner. The constructs of the present invention are useful for investigating gene functions, both physiologic and pathologic, in specific cell groups and/or at specific times. In addition, the technology of the present invention may be used in research and development to investigate both normal and disease pathways.

The present invention provides compositions for RNA interference and methods of use thereof. The present invention is based on the development of promoters that can be used to regulate shRNA expression spatially (in specific cells) and temporally (at specific times) in transgenic animals that express a recombinase. The compositions and methods of the present invention feature regulatable promoters that allow for inhibition of the expression of target alleles in a spatially and temporally regulatable manner. Thus, the compositions of the present invention are useful for investigating gene functions, both physiologic and pathologic, in specific cell groups and in specific ages, in normal and disease pathways. Functional and genomic and proteomic methods are featured. Therapeutic methods are also featured.

Other features and advantages of the invention will be apparent from the following detailed description, and from the claims.

### BRIEF DESCRIPTION OF THE DRAWINGS

FIGS. 1A-F depict various U6-shRNA transgenic constructs. (A) The basic shRNA expression construct. (B-D) Inducible desilencing constructs: the shRNA is expressed but upon exposure to Cre recombinase the expression will be inhibited. The Neo tail (B) is attached as a PCR marker for detection of transgenes and recombination events. (E, F) Inducible silencing constructs: the shRNA expression is blocked but upon exposure to Cre recombinase the expression will be activated.

FIGS. 2A-B depict the results of testing the U6 promoter modified by loxP site insertion. (A) SOD1^{G93A}GFP expression was quantified in cell lysates by emission scanning using a fluorometer (n=3) according to a published protocol (Chiu et al. 2002). Cotransfected constructs are indicated in the figure legend. (B) Detection of the transfected SOD1^{G93A}GFP and the endogenous human SOD1 proteins in 293 cells by Western blot.

FIG. 3 depicts ubiquitin C promoter-shRNA regulatable constructs.

FIG. 4 depicts the results of testing the tetracycline regulatable expression vectors UbCP-TRE1-EGFP and UbCP-TRE2-EGFP upon cotransfection with a vector coding for tTS.

FIG. 5 depicts the results of testing the mirMSOD2-expressing tetracycline regulatable expression vector UbCP-TRE2-mirMSOD2-tTS-IRES-EGFP following transfection into cells and addition of doxicyclin.

FIG. 6 depicts the structure of the mirMSOD2 shRNA (SEQ ID NO: 1).

FIG. 7 depicts the results of testing the tetracycline-regulatable mirMSOD2-expressing vector UbCP-TRE2-mirMSOD2-tTS-IRES-EGFP, and the cre-lox-regulatable mirMSOD2-expressing vector UbCP-lox-RFP-lox-mirMSOD2-EGFP, for their ability to inhibit endogenous SOD2 gene expression when transfected into cells and the cells are additionally exposed to doxicyclin or cre, respectively.

### DETAILED DESCRIPTION OF THE INVENTION

The present invention features regulatable constructs for the expression of shRNAs. In particular, the invention features constructs that can mediate RNAi (or gene silencing) in a spatial or temporal fashion. Spatial or temporal regulation is achieved by coexpressing a construct of the invention in a cell or animal which expresses a recombinase. The featured constructs of the invention are capable of being switched from an inactive to active form (or vice versa) upon interaction with the appropriate recombinase. Exemplary constructs comprise loxP sites which are present in certain orientations such that interaction with the recombinase, Cre, switches the constructs from inactive to active forms (or vice versa). Further featured constructs comprise tetracycline responsive elements. Regulation of shRNA expression is achieved by coexpressing these constructs in a cell or animal which expresses a tetracycline transcriptional repressor, and upon addition of an inducer (e.g., tetracycline or a tetracycline analog), allows the construct to switch from inactive to active forms.

In one aspect, the invention features inducible desilencing constructs. These constructs have been designed such that they express shRNA against a target gene, but upon exposure to Cre recombinase the shRNA-encoding sequences are excised, thereby stopping the shRNA expression and its silencing of target gene expression. In another aspect, the invention features inducible silencing constructs. These constructs do not express shRNA, but upon exposure to Cre recombinase or to tetracycline (or tetracycline analog), the construct activates and expresses the shRNA, thereby silencing target gene expression. In various embodiments, the shRNA encoding sequence is under the transcriptional control of a Pol III or Pol II promoter.

The present invention provides compositions for RNA interference and methods of use thereof. The present invention is based on the development of promoters, e.g., Pol III and Pol II promoters, that can be used to regulate shRNA expression spatially (in specific cells) and temporally (at specific times) in transgenic animals that express a recombinase. The compositions and methods of the present invention feature regulatable promoters that allow for inhibition of the expression of target alleles in a spatially and temporally regulatable manner. Thus, the compositions of the present invention are useful for investigating gene functions, both physiologic and pathologic, in specific cell groups and in specific ages, in normal and disease pathways. Functional and genomic and proteomic methods are featured. Therapeutic methods are also featured.

Accordingly, the invention features, in one aspect, a construct comprising a U6 promoter operably linked to a shRNA encoding nucleic acid sequence, the construct further comprising a first loxP site upstream of the promoter and a second loxP site downstream of the shRNA encoding sequence, the loxP sites being in the same orientation such that the promoter and encoding sequences are excisable upon exposure to Cre.

The invention features, in another aspect, a construct comprising a U6 promoter operably linked to a shRNA encoding nucleic acid sequence, the shRNA encoding sequence comprising a first stem-encoding portion, a loop-encoding portion, and a second stem-encoding portion, wherein the construct further comprises spacer DNA downstream of the shRNA encoding sequence, a second loxP site downstream of the spacer DNA, and a first loxP site within the loop-encoding portion of the shRNA encoding sequence, the loxP sites being in the same orientation such that the spacer DNA and second stem-encoding sequence are excisable upon exposure to Cre.

The invention features, in another aspect, a construct comprising a U6 promoter operably linked to a shRNA encoding nucleic acid sequence, the U6 promoter comprising (a) a distal sequence element (DSE); (b) a proximal sequence element (PSE); and (b) a TATA box, operably linked, wherein the construct further comprises a first loxP site downstream of the shRNA encoding sequence, and a second loxP site between the DSE and the PSE, the loxP sites being in the same orientation such that the shRNA encoding sequences and a portion of the promoter comprising the PSE and the TATA box are excisable upon exposure to Cre.

The invention features, in yet another aspect, a construct comprising a U6 promoter operably linked to a shRNA encoding nucleic acid sequence, the shRNA encoding sequence comprising a first stem-encoding portion, a loop-encoding portion, and a second stem-encoding portion, the loop-encoding portion comprising a first loxP site operably linked to a transcription termination signal upstream of a spacer DNA and a second loxP site, the loxP sites being in the same orientation such that the first loxP site, termination signal and spacer DNA are excisable upon exposure to Cre.

The invention features, in yet another aspect, a construct comprising a U6 promoter operably linked to a shRNA encoding nucleic acid sequence, the U6 promoter comprising (a) a distal sequence element (DSE); (b) a proximal sequence element (PSE); and (b) a TATA box, operably linked, wherein the construct further comprises a first loxP site and a second loxP site, said sites being interrupted by spacer DNA, between the DSE and the PSE, the loxP sites being in the same orientation such that a loxP site and the spacer DNA are excisable upon exposure to Cre.

The invention further features, in one aspect, an inducible desilencing construct for the expression of a shRNA, the construct comprising a promoter element operably linked to a shRNA encoding element and further comprising a first and second recombinase-sensitive element in an appropriate orientation such that all or a portion of the promoter or shRNA encoding element is excisable upon exposure to Cre.

In one embodiment of this aspect, the first recombinase-sensitive element is upstream of the promoter and the second recombinase-sensitive element is downstream of the shRNA encoding element.

In another embodiment of this aspect, the first recombinase-sensitive element is within the shRNA encoding element and the second recombinase-sensitive element is downstream of the shRNA encoding element. In one embodiment, the first element is within a loop portion of the shRNA encoding element. In another embodiment, the construct further includes a spacer nucleotide sequence between the shRNA encoding element and the second recombinase-sensitive element. In a preferred embodiment, the spacer is between 50 and 200 nucleotides in length.

In yet another embodiment of this aspect, the first recombinase-sensitive element is within the promoter and the second recombinase-sensitive element is downstream of the shRNA encoding element. In preferred embodiments, the first recombinase-sensitive element is downstream of at least one obligatory element in said promoter or is downstream of a DSE element.

The invention features, in one aspect, an inducible silencing construct for the expression of a shRNA, the construct comprising a promoter element operably linked to a shRNA encoding element, the promoter or shRNA encoding element being interrupted by DNA sequences flanked by a first and second recombinase-sensitive element in an appropriate orientation such that all or a portion of the DNA sequences is excisable upon exposure to Cre.

In one embodiment of this aspect, the DNA sequences flanked by a first and second recombinase-sensitive element are within the promoter. In one embodiment, the promoter is a Pol III promoter. In a preferred embodiment, the promoter is U6 comprising a distal sequence element (DSE), proximal sequence element (PSE) and TATA box. In one embodiment, the DNA sequences flanked by a first and second recombinase-sensitive element are between the DSE and PSE.

In another embodiment of this aspect, the DNA sequences flanked by a first and second recombinase-sensitive element are within the shRNA encoding element. In one embodiment, the DNA sequences flanked by a first and second recombinase-sensitive element comprise a transcription termination signal.

In embodiments of this aspect, the construct further includes a spacer nucleotide sequence between the first recombinase-sensitive element and the second recombinase-sensitive element. Preferably, the spacer is between about 50 and 200 nucleotides in length.

The invention features, in one aspect, an inducible silencing construct for the expression of a shRNA, the construct comprising a ubiquitin C promoter (UbC) operably linked to an intron comprising an shRNA encoding element, the Ubc promoter comprising a 5′ promoter region and exon 1, operably linked, wherein the construct further comprises one or more tetracycline responsive elements (TRE) within the 5′ promoter region or exon 1.

In one embodiment of this aspect, the construct further comprises a tetracycline transcriptional repressor (tTs) encoding nucleic acid sequence and an internal ribosomal entry site (IRES). In various embodiments, the constructs further comprise a marker protein encoding nucleic acid sequence.

The invention features, in another aspect, an inducible silencing construct for the expression of a shRNA, the construct comprising a ubiquitin C promoter (UbCP) operably linked to an intron, the Ubc promoter comprising a 5′ promoter region and exon 1, operably linked, wherein the intron comprises an shRNA encoding element downstream of a transcription termination signal, and wherein the construct further comprises a first loxP site in said exon 1, and a second loxP site between the transcription termination signal and the shRNA encoding element, the loxP sites being in the same orientation such that a portion of the intron comprising the transcription termination signal is excisable upon exposure to Cre.

In one embodiment of this aspect, the construct further comprises a marker protein encoding nucleic acid sequence upstream of the transcription termination site. In various embodiments, the constructs further comprise a marker protein nucleic acid sequence downstream of the shRNA encoding element.

The invention features, in yet another aspect, an inducible desilencing construct for the expression of a shRNA, the construct comprising a ubiquitin C promoter (UbCP) operably linked to an intron, the Ubc promoter comprising a 5′ promoter region and exon 1, operably linked, wherein the intron comprises an shRNA encoding element upstream of a transcription termination signal, and wherein the construct further comprises a first loxP site in said exon 1, and a second loxP site downstream of the transcription termination signal, the loxP sites being in the same orientation such that a portion of the intron comprising the shRNA encoding element and the transcription termination signal is excisable upon exposure to Cre.

In one embodiment of this aspect, the construct further comprises a marker protein encoding nucleic acid sequence between the shRNA encoding element and the transcription termination signal. In various embodiments, the constructs further comprise a marker protein encoding nucleic acid sequence downstream of the second loxP site.

In particular embodiments of these aspects, the marker protein is red or green fluorescent protein.

In one embodiment of these aspects, the recombination-sensitive element is a loxP site.

In one embodiment of these aspects, the shRNA comprises a sequence sufficiently complementary to a target mRNA to mediate degradation of said target. In a preferred embodiment, the target mRNA encodes a mutant protein. Preferably, the mutant protein is a disease-causing mutant, e.g., SOD1. In certain embodiments, the mutant protein is SOD1^{G93A} or SOD1^{G85R}.

The invention further features a construct of any one of these aspects for the treatment of a disease. In one embodiment, the disease is caused by aberrant gene function. In a preferred embodiment, the disease is a dominant, gain-of-function mutation. In one embodiment, the disease is a neurological disease.

The invention also features a vector comprising the construct of any one of these aspects. In one embodiment, the vector is a viral vector, e.g., an AAV or lentivirus.

The invention further features a cell comprising a construct or a vector of any one of these aspects. In a preferred embodiment, the cell is an animal cell.

The invention still further features a nonhuman transgenic animal carrying a transgene comprising the constructs of any one of these aspects. The invention also provides a nonhuman homologous recombinant animal which contains cells from any one of these aspects.

The invention features, in another aspect, a method for promoting inducible RNAi comprising introducing into a cell a construct of the invention under conditions such that shRNA expression is inducible.

In one embodiment, the cell is present in a subject. In another embodiment, the cell is a cultured cell.

In one embodiment, the introducing comprises transfecting said cell. In another embodiment, the introducing comprises infecting said cell with a viral vector.

The invention features, in one aspect, a method of promoting inducible RNAi in a subject, the method comprising administering a construct of the invention.

The invention features, in one aspect, a method for selectively inhibiting mutant gene expression in vivo or in vitro, the method comprising introducing into a host cell a construct of the invention under conditions such that said shRNA is expressed, thereby inhibiting mutant gene expression.

In one embodiment, the shRNA does not inhibit expression of the wild type allele.

The invention features, in another aspect, a method for treating a disease in a subject, the method comprising administering a construct of the invention, thereby treating a disease in a subject.

In one embodiment, the disease is caused by aberrant gene function. In one embodiment, the disease is caused by a mutation that is a dominant, gain-of-function mutation.

The invention features, in one aspect, a method for identifying a compound which modulates RNAi, the method comprising contacting a cell comprising a construct of the invention with a test compound, and determining the effect of the test compound on an indicator of RNAi activity in said cell, thereby identifying a compound which modulates RNAi.

The invention provides, in a related aspect, a compound identified according to the above method.

The invention features, in yet another related aspect, a method for modulating RNAi, the method comprising contacting a cell expressing a construct of the invention with a compound that modulates RNAi, as identified according to the above method, in a sufficient concentration to modulate the activity of RNAi.

The invention further provides a method for modulating RNAi, the method comprising contacting a cell expressing a construct of the invention with a compound which binds to said construct in a sufficient concentration to modulate the activity of RNAi.

The invention provides, in another aspect, a method for deriving information about the function of a gene in a cell or organism comprising introducing into said cell or organism a construct of the invention; maintaining the cell or organism under conditions such that RNAi can occur; determining a characteristic or property of said cell or organism; and comparing said characteristic or property to a suitable control, the comparison yielding information about the function of the gene.

The invention features, in one aspect, a method of validating a candidate protein as a suitable target for drug discovery comprising: (a) introducing into a cell or organism z construct of the invention; (b) maintaining the cell or organism under conditions such that RNAi can occur; (c) determining a characteristic or property of said cell or organism; and (d) comparing said characteristic or property to a suitable control, the comparison yielding information about whether the candidate protein is a suitable target for drug discovery.

The invention features, in another aspect, a kit comprising reagents for activating RNAi in a cell or organism, said kit comprising: a construct of the invention and instructions for use.

The invention features, in yet another aspect, a method of excising a DNA sequence, the method comprising exposing a construct of the invention to Cre recombinase and allowing recombination, thereby excising a portion of said DNA sequence.

The invention features, in still another aspect, a method of promoting target gene expression, the method comprising exposing a construct of the invnetion to a Cre recombinase; excising of a portion of the shRNA flanked by loxP sites; and disrupting expression of the shRNA, thereby allowing the target gene to be expressed.

In one embodiment of this aspect, said disrupted expression results in the silencing of a mutant gene. In a preferred embodiment, the mutant gene is SOD 1.

The invention features, in one aspect, a method of recovering promoter function, the method comprising exposing a construct of the invention to a Cre recombinase protein under conditions such that shRNA expression is activated, thereby recovering said promoter function.

The invention features, in another aspect, a method of disrupting promoter function, the method comprising exposing a construct of the invention to a Cre recombinase, and allowing recombination, thereby disrupting promoter function.

The invention features, in yet another aspect, a method of inhibiting expression of a target gene, the method comprising exposing a construct of the invention to a Cre recombinase; activating said promoter; and expressing said shRNA, thereby inhibiting target gene expression.

In one embodiment, the promoter is regulated in an animal. In one embodiment, the promoter is regulated temporally. In another embodiment, the promoter is regulated spatially.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

I. Definitions

So that the invention may be more readily understood, certain terms are first defined.

As used herein, the term “Cre” has its art-recognized meaning, i.e., the enzyme expression product of the cre gene which is a recombinase that effects site-specific recombination of DNA at lox sites. A preferred cre gene is the bacteriophage PI cre gene as disclosed, for example, in Abremski et al., Cell, 32:1301-1311 (1983), the entire content of which is incorporated herein by reference.

As used herein, the term “lox site” or “loxP site” has its art known meaning, e.g., a nucleotide sequence at which the gene product of the cre gene, referred to herein as “Cre,” can catalyze a site-specific recombination. A LoxP site is a 34 base pair nucleotide sequence which can be isolated from bacteriophage PI by methods known in the art.

As used herein the term “site-specific recombination” refers to a recombination event that is effected between two specific sites on a single nucleic acid molecule or between two different molecules that requires the presence of an exogenous protein, such as an integrase or recombinase. “Cre-lox site-specific recombination” refers to deletion of a pre-selected DNA segment flanked by lox sites. The term “DNA segment” refers to a linear fragment of single- or double-stranded deoxyribonucleic acid (DNA), which can be derived from any source.

As used herein, the term “encodes” means the generation of a RNA molecule from a DNA molecule (i.e., a complementary RNA molecule generated from the DNA molecule by the process of transcription) or the generation of a polypeptide or protein molecule from a DNA molecule via a RNA intermediate (i.e., by the processes of transcription and translation).

The term “construct”, as used herein refers to an engineered DNA molecule including one or more nucleotide sequences from different sources. A preferred construct includes at least a shRNA-encoding region operably linked to a promoter sequence.

The term “enhancer” refers to a DNA sequence which, when bound by a specific protein factor, enhances the levels of expression of a gene, but is not sufficient alone to cause expression. An “enhancer” is capable of enhancing expression of a gene regardless of the distance from the gene or orientation relative to the gene.

The term “kit” is any manufacture (e.g. a package or container) comprising at least one reagent, e.g. a construct, for activating RNAi in a cell or organism, the manufacture being promoted, distributed, or sold as a unit for performing the methods of the present invention.

The term “intron” refers to a sequence that is not translated into protein. An intron is initially transcribed into RNA but is cut out of the message before it is translated into protein.

The term “gene” includes cDNAs, RNA, or other polynucleotides that encode gene products. “Foreign gene” denotes a gene that has been obtained from an organism or cell type other than the organism or cell type in which it is expressed; it also refers to a gene from the same organism that has been translocated from its normal situs in the genome.

The term “target gene”, as used herein, refers to a gene intended for downregulation via RNA interference (“RNAi”). The term “target protein” refers to a protein intended for downregulation via RNAi. The term “target RNA” refers to an RNA molecule intended for degradation by RNAi. An exemplary “target RNA” is a coding RNA molecule (i.e., a mRNA molecule).

The term “promoter” refers to a DNA sequence to which RNA polymerase can bind and initiate transcription. An “inducible promoter” is a DNA sequence which, when operably linked with a DNA sequence encoding a specific gene product, causes the gene product to be substantially produced in a cell only when an inducer which corresponds to the promoter is present in the cell. The term “Pol III promoter” refers to an RNA polymerase III promoter. Exemplary Pol III promoters include, but are not limited to, the U6 promoter, the H1 promoter, and the tRNA promoters. The term “Pol II promoter” refers to an RNA polymerase II promoter. Exemplary Pol II promoters include, but are not limited to, the Ubiquitin C promoter and the CMV promoter.

The term “inducible RNAi” refers to RNAi-mediated silencing that can be regulated, e.g., spatially or temporally. Inducible RNAi is intended to encompass both inducible silencing, e.g., effecting, promoting or stimulating RNAi-mediated silencing, and inducible desilencing, e.g., inhibiting or downmodulating of RNAi-mediated silencing.

The term an “inducible desilencing construct” refers to a DNA sequence which is capable of expressing a shRNA, but upon the occurrence of a site-specific recombinase mediated event, no longer expresses the shRNA. Upon exposure to a recombinase, e.g., Cre, the DNA sequence, e.g., a portion of the DNA sequence containing the promoter or shRNA encoding element, may be excised, thereby preventing shRNA expression and its silencing of a target gene expression.

The term an “inducible silencing construct” refers to a DNA sequence which is capable of expressing a shRNA only upon the occurrence of a site-specific recombinase mediated event. Upon exposure to a recombinase, e.g., Cre, the DNA sequence, e.g., the promoter or shRNA encoding element (e.g., the transgene), is activated such that the shRNA is expressed, thereby silencing expression of a target gene.

The term “expression” of a gene or nucleic acid encompasses not only cellular gene expression, but also the transcription and translation of nucleic acid(s) in cloning systems and in any other context. The term “recombinase” encompasses enzymes that induce, mediate or facilitate recombination, and other nucleic acid modifying enzymes that cause, mediate or facilitate the rearrangement of a nucleic acid sequence, or the excision or insertion of a first nucleic acid sequence from or into a second nucleic acid sequence.

The term “RNA interference” or “RNAi”, as used herein, refers generally to a sequence-specific or selective process by which a target molecule (e.g., a target gene, protein or RNA) is downregulated. In specific embodiments, the process of “RNA interference” or “RNAi” features degradation of RNA molecules, e.g., RNA molecules within a cell, said degradation being triggered by an RNA agent. Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

The term “RNA agent”, as used herein, refers to an RNA (or analog thereof), comprising a sequence having sufficient complimentarity to a target RNA (i.e., the RNA being degraded) to direct RNAi. A sequence having a “sufficiently complementary to a target RNA sequence to direct RNAi” means that the RNA agent has a sequence sufficient to trigger the destruction of the target RNA by the RNAi machinery (e.g., the RISC complex) or process.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double-stranded, i.e., dsRNA and dsDNA, respectively).

The term “mRNA” or “messenger RNA” refers to a single-stranded RNA that specifies the amino acid sequence of one or more polypeptide chains. This information is translated during protein synthesis when ribosomes bind to the mRNA.

The term “target site” of a recombinase is the nucleic acid sequence or region that is recognized (e.g., specifically binds to) and/or acted upon (excised, cut or induced to recombine) by the recombinase. The term “gene product” refers primarily to proteins and polypeptides encoded by other nucleic acids (e.g., non-coding and regulatory RNAs such as tRNA, sRNPs). The term “regulation of expression” refers to events or molecules that increase or decrease the synthesis, degradation, availability or activity of a given gene product.

The term “transcript” refers to a RNA molecule transcribed from a DNA or RNA template by a RNA polymerase template. The term “transcript” includes RNAs that encode polypeptides (i.e., mRNAs) as well as noncoding RNAs (“ncRNAs”).

As used herein, the term “small interfering RNA” (“siRNA”) (also referred to in the art as “short interfering RNAs”) refers to an RNA agent, preferably a double-stranded agent, of about 10-50 nucleotides in length (the term “nucleotides” including nucleotide analogs), preferably between about 15-25 nucleotides in length, more preferably about 17, 18, 19, 20, 21, 22, 23, 24, or 25 nucleotides in length, the strands optionally having overhanging ends comprising, for example, 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., the RISC complex).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

The term “subject”, as used herein, includes living organisms at risk for or having a cell neurological, e.g. neurodegenerative disease or disorder. Examples of subjects include humans, monkeys, cows, sheep, goats, dogs, cats, mice, rats, and transgenic species thereof. Administration of the compositions of the present invention to a subject to be treated can be carried out using known procedures, at dosages and for periods of time effective to modulate RNAi in the subject as further described herein.

The term “treatment”, as used herein, is defined as the application or administration of a therapeutic agent to a subject, or application or administration of a therapeutic agent to an isolated tissue or cell line from a subject, who has a disease or disorder, a symptom of a disease or disorder, or a predisposition toward a disease or disorder, with the purpose to cure, heal, alleviate, relieve, alter, remedy, ameliorate, improve or affect the disease or disorder, the symptoms of the disease or disorder, or the predisposition toward a disease or disorder. A therapeutic agent includes, but is not limited to, small molecules, peptides, antibodies, ribozymes, antisense oligonucleotides, chemotherapeutic agents and radiation.

The term “effective amount”, as used here in, is defined as that amount necessary or sufficient to treat or prevent a disorder, e.g. a neurological or a neurodegenerative disease or disorder. The effective amount can vary depending on such factors as the size and weight of the subject, the type of illness, or the particular agent being administered. One of ordinary skill in the art would be able to study the aforementioned factors and make the determination regarding the effective amount of the agent without undue experimentation.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “mutation” refers to a substitution, addition, or deletion of a nucleotide within a gene sequence resulting in aberrant production (e.g., misregulated production) of the protein encoded by the gene sequence. A “gain-of-function” mutation is a mutation that results in production of a protein having aberrant function as compared to the wild-type or normal protein encoded by a gene sequence.

The term “pharmaceutical composition” as used herein, refers to an agent formulated with one or more compatible solid or liquid filler diluents or encapsulating substances which are suitable for administration to a human or lower animal.

A gene “involved” in a disorder includes a gene, the normal or aberrant expression or function of which effects or causes a disease or disorder or at least one symptom of said disease or disorder.

The phrase “examining the function of a gene in a cell or organism” refers to examining or studying the expression, activity, function or phenotype arising therefrom.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNAi agent of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

The term “upstream” refers to nucleotide sequences that precede, e.g., are on the 5′ side of, a reference sequence.

The term “downstream” refers to nucleotide sequences that follow, e.g., are on the 3′ side of, a reference sequence.

The terms used herein are not intended to be limiting of the invention.

II. shRNA-Encoding Nucleic Acids

Preferred constructs of the instant invention include nucleic acid sequences or molecules that encode (i.e., generate) shRNA molecules. The requisite elements of a shRNA-encoding nucleic acid sequence or molecule include a first portion and a second portion, having sequences such that the RNA sequences encoded by said portions have sufficient complementarity to anneal or hybridize to form a duplex or double-stranded stem portion. The two portions need not be fully or perfectly complementary. The first and second “stem-encoding” portions are connected by a portion having a sequence that, when encoded, has insufficient sequence complementarity to anneal or hybridize to other portions of the shRNA. This latter portion is referred to as a “loop-encoding” portion in the shRNA-encoding nucleic acid sequences or molecules. The shRNA-encoding nucleic acid sequences or molecules are transcribed to generate shRNAs. shRNAs can also include one or more bulges, i.e., extra nucleotides that create a small nucleotide “loop” in a portion of the stem, for example a one-, two- or three-nucleotide loop. The encoded stem portions can be the same length, or one portion can include an overhang of, for example, 1-5 nucleotides. The overhanging nucleotides can include, for example, uracils (Us), e.g., all Us. Such Us are notably encoded by thymidines (Ts) in the shRNA-encoding DNA which signal the termination of transcription.

One strand of the stem portion of the encoded shRNA is further sufficiently complementary (e.g., antisense) to a target RNA (e.g., mRNA) sequence to mediate degradation or cleavage of said target RNA via RNA interference (RNAi). The antisense portion can be on the 5′ or 3′ end of the stem. The stem-encoding portions of a shRNA-encoding nucleic acid (or stem portion of a shRNA) are preferably about 15 to about 50 nucleotides in length. When used in mammalian cells, the length of the stem portions should be less than about 30 nucleotides to avoid provoking non-specific responses like the interferon pathway. In non-mammalian cells, the stem can be longer than 30 nucleotides. In fact, a stem portion can include much larger sections complementary to the target mRNA (up to, and including the entire mRNA). The loop portion in the shRNA (or loop-encoding portion in the encoding DNA) can be about 2 to about 20 nucleotides in length, i.e., about 2, 3, 4, 5, 6, 7, 8, 9, or more, e.g., 15 or 20, or more nucleotides in length. A preferred loop consists of or comprises a “tetraloop” sequences. Exemplary tetraloop sequences include, but are not limited to, the sequences GNRA, where N is any nucleotide and R is a purine nucleotide, GGGG, and UUUU.

The sequence of the antisense portion of a shRNA can be designed by selecting an 18, 19, 20, 21 nucleotide, or longer, sequence from within the target RNA (e.g., mRNA), for example, from a region 100 to 200 or 300 nucleotides upstream or downstream of the start of translation. In general, the sequence can be selected from any portion of the target RNA (e.g., mRNA) including the 5′ UTR (untranslated region), coding sequence, or 3′ UTR. This sequence can optionally follow immediately after a region of the target gene containing two adjacent AA nucleotides. The last two nucleotides of the nucleotide sequence can be selected to be UU. shRNAs so generated are processed under appropriate conditions (e.g., in an appropriate in vitro reaction or in a cell) by RNAi machinery (i.e., Dicer and/or RISC complexes) to generate siRNAs. shRNAs can be synthesized exogenously or can be transcriped in vivo from an RNA polymerase (e.g., a Pol II or Pol III polymerase), thus permitting the construction of continuous cell lines or transgenic animals in which the desired gene silencing is stable and heritable.

In certain aspects of the invention, it may be important to detect the generation or expression of shRNAs, target mRNAs and/or the gene products encoded by said target RNAs. The detection methods used herein include, for example, cloning and sequencing, ligation of oligonucleotides, use of the polymerase chain reaction and variations thereof (e.g., a PCR that uses 7-deaza GTP), use of single nucleotide primer-guided extension assays, hybridization techniques using target-specific oligonucleotides that can be shown to preferentially bind to complementary sequences under given stringency conditions, and sandwich hybridization methods.

Sequencing may be carried out with commercially available automated sequencers utilizing labeled primers or terminators, or using sequencing gel-based methods. Sequence analysis is also carried out by methods based on ligation of oligonucleotide sequences which anneal immediately adjacent to each other on a target DNA or RNA molecule (Wu and Wallace, Genomics 4: 560-569 (1989); Landren et al., Proc. Natl. Acad. Sci. 87: 8923-8927 (1990); Barany, F., Proc. Natl. Acad. Sci. 88: 189-193 (1991)). Ligase-mediated covalent attachment occurs only when the oligonucleotides are correctly base-paired. The Ligase Chain Reaction (LCR), which utilizes the thermostable Taq ligase for target amplification, is particularly useful for interrogating late onset diabetes mutation loci. The elevated reaction temperatures permits the ligation reaction to be conducted with high stringency (Barany, F., PCR Methods and Applications 1: 5-16 (1991)).

The hybridization reactions may be carried out in a filter-based format, in which the target nucleic acids are immobilized on nitrocellulose or nylon membranes and probed with oligonucleotide probes. Any of the known hybridization formats may be used, including Southern blots, slot blots, “reverse” dot blots, solution hybridization, solid support based sandwich hybridization, bead-based, silicon chip-based and microtiter well-based hybridization formats.

Detection oligonucleotide probes range in size between 10-1,000 bases. In order to obtain the required target discrimination using the detection oligonucleotide probes, the hybridization reactions are generally run between 20°-60° C., and most preferably between 30°-50° C. As known to those skilled in the art, optimal discrimination between perfect and mismatched duplexes is obtained by manipulating the temperature and/or salt concentrations or inclusion of formamide in the stringency washes.

Detection of proteins may be carried out using specific antibodies, e.g., monoclonal or polyclonal antibodies, or fragments thereof.

Preferred detection reagents are labeled, e.g., fluorescents, coloro-metrically or radio-iso-typically labeled to facilitate visulalization and/or quantitation.

III. Regulatable Systems

A. Regulation of shRNA Expression by Site-Specific Recombinase System

The present invention provides a regulatory system which utilizes a site specific recombinase system to regulate shRNA expression in eukaryotic cells. In particular, this invention provides recombinase-regulated shRNA expression constructs comprising an shRNA encoding nucleic acid sequence and additionally comprising two recombinase-sensitive elements in an appropriate orientation such that all or a portion of the construct is excisable upon exposure to Cre. In one embodiment, the shRNA encoding nucleic acid sequence is under the control of a Pol III promoter, e.g., U6 promoter. In another embodiment, the shRNA encoding nucleic acid sequence is under the control of a Pol II promoter, e.g., Ubiquitin C promoter (UbCP). The construct may further comprise a marker protein encoding nucleic acid sequence in order to follow shRNA expression. The marker protein can be any marker protein commonly known in the art, e.g., red fluorescent protein or green fluorescent protein.

The present invention utilizes a site specific recombinase system, e.g., the Cre/lox system of bacteriophage P1, which is widely used to engineer gene recombination in mice and plants. Other different site specific recombinase systems that may be used include the FLP/FRT system of yeast (see e.g., O'Gormen et al. (1991) Science 251:1351), the Gin recombinase of phage Mu, the Pin recombinase of E. coli, and the R/RS system of the pSR1 plasmid. In the Cre/lox system, a recombinase protein, e.g., the Cre recombinase protein, will interact specifically with its respective site-specific recombination sequence, known as lox sites, to invert or excise the intervening sequences.

Cre is a 38 kDa recombinase protein from bacteriophage PI which mediates intramolecular (excisive or inversional) and intermolecular (integrative) site specific recombination between loxP sites (see review article by Brian Sauer in Methods of Enzymology; 1993, Vol. 225, 890-900). The constructs of the instant invention feature loxP sites placed in an orientation such that excisive site specific recombination occurs. A loxP site (locus of crossing over) consists of two 13 bp inverted repeats separated by an 8 bp asymmetric spacer region. Preferably, the two loxP sites are placed at least about 10, 50, 100, 150, 200, 300, 400, 500 or more nucleotides apart. More preferably, the two loxP sites are placed at least about 10, 20, 30, 40, 50, 60, 70, 80, 90, or 100 nucleotides apart. Even more preferably, the two loxP sites are placed at least 60, 65, 70, 75, 80, 85, 90, 95 or 100 nucleotides apart. Most preferably, the two loxP sites are placed at least 80, 81, 82, 83, 84, 85, 86, 87, 88, 89, 90 or more nucleotides apart.

The nucleotide sequence of the insert repeats and the spacer region of LoxP is as follows: ATAACTTCGTATA ATGTATGC TATACGAAGTTAT. Other suitable lox sites include LoxB, LoxC2, LoxL and LoxR sites which are nucleotide sequences isolated from E. coli. These sequences are disclosed and described by Hoess et al., Proc. Natl. Acad. Sci. USA 79:3398 (1982), the entire disclosure of which is hereby incorporated herein by reference. Preferably, the lox site is LoxP.

One molecule of Cre binds per inverted repeat or two Cre molecules line up at one loxP site. The recombination occurs in the asymmetric spacer region. Those 8 bases are also responsible for the directionality of the site. Two loxP sequences in direct orientation dictate excision of the intervening DNA between the sites leaving one loxP site behind.

In particular, recombination between lox sites in the same orientation results in a deletion of the DNA segment located between the two lox sites and a connection between the resulting ends of the original DNA molecule. The deleted DNA segment forms a circular molecule of DNA. The original DNA molecule and the resulting circular molecule each contain a single lox site.

B. Regulation of shRNA Expression by Tetracycline or Analogues thereof

The present invention provides a regulatory system which utilizes components of the Tet repressor/operator/inducer system of prokaryotes to regulate shRNA expression in eukaryotic cells. In particular, this invention provides tetracycline-regulated shRNA expression constructs comprising an shRNA encoding nucleic acid sequence linked to one or more tet operator sequences. In one embodiment, the shRNA encoding nucleic acid sequence is under the control of a Pol II promoter, e.g., Ubiquitin C promoter (UbCP). However, it will be understood that a Pol III promoter is also contemplated. The constructs may additionally comprise nucleotide sequences encoding a tetracycline transcriptional inhibitor or tetracycline transcriptional activator. In one embodiment, the nucleotide sequences encoding a tetracycline transcriptional inhibitor or activator are under the transcriptional control of the same Pol II promoter, e.g., Ubiquitin C promoter. In another embodiment, the nucleotide sequences encoding a tetracycline transcriptional inhibitor or activator are under the transcriptional control of an independent Pol II promoter. Alternatively, the tetracycline inhibitor or activator may be present and introduced into host cells on a second, distinct expression vector or may be stable integrated into the host cell's genome. The tetracycline-regulated shRNA expression constructs are useful in methods of modulating or regulating shRNA expression in a highly controlled manner.

This invention provides expression constructs encoding the shRNA operatively linked to one or more tet operator sequences, and in certain embodiments, additionally encoding the transcriptional repressor or activator. In a preferred embodiment, the shRNA is operatively linked to at least two or more let operator sequences. The invention further provides recombinant expression vectors containing these constructs in a form suitable for expression of the encoded shRNA and transcriptional repressor or activator in a host cell. The invention still further provides host cells into which a recombinant expression vector of the invention has been introduced. Thus, an shRNA is expressed in these host cells. The host cell can be, for example, a mammalian cell (e.g., a human cell), a yeast cell, a fungal cell or an insect cell. Moreover, the host cell can be a fertilized non-human oocyte, in which case the host cell can be used to create a transgenic organism having cells that express the shRNA. Still further, the recombinant expression vector can be designed to allow homologous recombination between the nucleic acid encoding the shRNA and the genomic DNA in a host cell. Such homologous recombination vectors can be used to create homologous recombinant animals that express an shRNA of the invention.

To regulate transcription of the let operator-linked shRNA in these host cells, the concentration of Tc (or analogue thereof) in contact with the host cell is altered. For example, when a tetracycline transcriptional repressor binds to tet operator sequences in the absence of Tc, the concentration of Tc in contact with the cells is increased to thereby induce transcription of the let operator-linked shRNA. Alternatively, when the tetracycline transcriptional repressor binds to let operator sequences in the presence of Tc, the concentration of Tc in contact with the cells is decreased to thereby induce transcription of the let operator-linked shRNA.

In one aspect of the invention, shRNA expression is controlled by using a tetracycline transcriptional repressor protein that binds to let operator sequences. Such tetracycline-based regulatory systems are well described and will be well known to one of skill in the art. In one embodiment, shRNA expression can be controlled by using the tetracycline transcriptional repressor. Alternatively, shRNA expression can be controlled by using a transcriptional inhibitor fusion protein. A transcriptional inhibitor fusion protein may be composed of at least two polypeptides, a first polypeptide that binds to let operator sequences, e.g., a tetracycline transcriptional repressor, and a heterologous second polypeptide that directly or indirectly inhibits transcription in eukaryotic cells. The heterologous second polypeptide is derived from a different protein than the first polypeptide. Because the fusion proteins include a eukaryotic transcriptional silencer domain, they are anticipated to be more efficient at repressing transcription in eukaryotic cells than is a Tet Repressor alone.

Where a transcriptional inhibitor fusion protein is employed, in one embodiment the first polypeptide of the inhibitor fusion protein binds to tet operator sequences in the absence but not the presence of tetracycline (Tc) or an analogue thereof (e.g., the first polypeptide is preferably a Tet repressor). Thus, in the absence of tetracycline (or Tc analogue), this fusion protein binds to tet operator sequences operatively linked to shRNA encoding sequence, thereby inhibiting transcription of the shRNA. In another embodiment, the first polypeptide binds to tet operator sequences in the presence but not the absence of tetracycline (e.g., the first polypeptide is preferably a mutated Tet repressor). In the presence of tetracycline (or Tc analogue), this fusion protein binds to tet operator sequences operatively linked to an shRNA encoding sequence, thereby inhibiting transcription of the shRNA. The second polypeptide can be a transcriptional “silencer” domain from a protein such as the v-erbA oncogene product, the Drosophila Krueppel protein, the retinoic acid receptor alpha, the thyroid hormone receptor alpha, the yeast Ssn6/Tup1 protein complex, the Drosophila protein even-skipped, SIR1, NeP1, the Drosophila dorsal protein, TSF3, SFI, the Drosophila hunchback protein, the Drosophila knirps protein, WT1, Oct-2.1, the Drosophila engrailed protein, E4BP4 or ZF5. The fusion protein may further comprise additional polypeptides, such as a third polypeptide which promotes transport of the fusion protein into a cell nucleus (i.e., a nuclear transport amino acid sequence).

These methods can be used to regulate basal, constitutive or tissue-specific transcription and to induce transcription of a tetO-linked shRNA of interest. For example, an shRNA of interest that is operatively linked to one or more tetO sequences (e.g., two tetO sequences), and optionally additional positive regulatory elements (e.g., consitutive or tissue-specific enhancer sequences), will be transcribed in host cells at a level that is primarily determined by the strength of the positive regulatory elements in the host cell. Moreover, an shRNA of interest that is operatively linked to tetO sequences and only a minimal promoter sequence may exhibit varying degrees of basal level transcription depending on the host cell or tissue and/or the site of integration of the sequence. In a host cell containing such an shRNA encoding sequence and expressing a tetracycline transcriptional repressor or inhibitor fusion protein, transcription of the shRNA sequence can be upregulated in a controlled manner by altering (e.g., increasing) the concentration of Tc (or analogue) in contact with the host cell. For example, when the tetracycline transcriptional repressor or inhibitor fusion protein binds to tetO in the absence of Tc, the concentration of Tc in contact with the host cell is increased to induce expression of the shRNA. For example, Tc is added to the culture medium of a host cell or Tc is administered to a host organism to repress shRNA expression. Alternatively, when the inhibitor fusion protein binds to tetO in the presence of Tc, the concentration of Tc in contact with the host cell is decreased to induce expression of the shRNA.

The tetracycline-regulated shRNA expression constructs described herein can regulate a tetO-linked shRNA in which the tetO sequences are positioned 5′ of a minimal promoter sequence. Furthermore, tetracycline-regulated shRNA expression constructs described herein can regulate a tetO-linked shRNA in which tetO-linked sequences are located 3′ of the promoter sequence but 5′ of the transcription start site. Still further, tetracycline-regulated shRNA expression constructs described herein can regulate a tetO-linked shRNA in which tetO-linked sequences are located 3′ of the transcription start site.

It will be understood that, in the compositions and methods of the instant invention, shRNA expression can also be controlled by using a tetracycline transcriptional activator fusion protein. Such systems are well described and will be known to one of skill in the art. In a host cell which carries nucleic acid encoding a transcriptional activator fusion protein and an shRNA encoding nucleotide sequence operatively linked to the tet operator sequence (either on the same vector or two distinct vectors), high level transcription of the shRNA encoding nucleotide sequence operatively linked to the tet operator sequence(s) does not occur in the absence of the inducing agent, e.g., tetracycline or analogues thereof. The level of basal transcription of the nucleotide sequence may vary depending upon the host cell and site of integration of the sequence, but is generally quite low or even undetectable in the absence of Tc. In order to induce transcription in a host cell, the host cell is contacted with tetracycline or a tetracycline analogue. Accordingly, another aspect of the invention pertains to methods for stimulating transcription of an shRNA encoding nucleotide sequence operatively linked to one or more tet operator sequences in a host cell or animal which expresses a transcriptional activator fusion protein. The methods involve contacting the cell with tetracycline or a tetracycline analogue or administering tetracycline or a tetracycline analogue to a subject containing the cell.

In addition to regulating shRNA expression using either a transcriptional activator or inhibitor alone, the two types of proteins can be used in combination to allow for both positive and negative regulation of expression of one or more shRNA encoding sequences in a host cell. Thus, a transcriptional inhibitor protein that binds to tetO either (i) in the absence, but not the presence, of Tc, or (ii) in the presence, but not the absence, of Tc, can be used in combination with a transcriptional activator protein that binds to tetO either (i) in the absence, but not the presence, of Tc, or (ii) in the presence, but not the absence, of Tc.

For example, expression of a tetO-linked shRNA encoding sequence in a host cell is regulated in both a negative and positive manner by the combination of an inhibitor fusion protein that binds to tetO in the absence, but not the presence, of tetracycline or analogue thereof (referred to as a tetracycline controlled silencing domain, or tSD) and an activator fusion protein that binds to tetO in the presence, but not the absence, of tetracycline or analogue thereof (referred to as a reverse tetracycline controlled transactivator, or rtTA). In addition to tetO sequences, the shRNA encoding sequence is linked to a promoter (e.g., a Pol II promoter, e.g., a Ubiquitin C promoter), and may optionally contain other positive regulatory elements (e.g., enhancer sequences) that contribute to basal level, constitutive transcription of the shRNA in the host cell. Binding of tSD to the tetO sequences in the absence of tetracycline or analogue (e.g., doxycycline) inhibits the basal constitutive transcription of the shRNA encoding sequence, thus keeping the shRNA encoding sequence in a repressed state until shRNA expression is desired. When expression is desired, the concentration of tetracycline or analogue (e.g., doxycycline) in contact with the host cell increased. Upon addition of the drug, tSD loses the ability to bind to tetO sequences whereas the previously unbound rtTA acquires the ability to bind to tetO sequences. The resultant binding of rtTA to the tetO sequences linked to the shRNA encoding sequence thus stimulates transcription of the shRNA. The level of expression may be controlled by the concentration of tetracycline or analogue, the type of Tc analogue used, the duration of induction, etc., as described previously herein. It will be appreciated that the reverse combination of fusion proteins (i.e., the inhibitor binds in the presence but not the absence of the drug and the activator binds in the absence but not the presence of the drug) can also be used. In this case, expression of the shRNA encoding sequence is kept repressed by contacting the host cell with the drug (e.g., culture with Tc or analogue) and expression is activated by removal of the drug.

The term “tetracycline analogue” is intended to include compounds which are structurally related to tetracycline and which bind to the Tet repressor with a Kₐ of at least about 10⁶ M⁻¹. Preferably, the tetracycline analogue binds with an affinity of about 10⁹ M⁻¹ or greater. Examples of such tetracycline analogues include, but are not limited to, anhydrotetracycline, doxycycline, chlorotetracycline, oxytetracycline and others disclosed by Hlavka and Boothe, “The Tetracyclines,” in Handbook of Experimental Pharmacology 78, R. K. Blackwood et al. (eds.), Springer-Verlag, Berlin-New York, 1985; L. A. Mitscher, “The Chemistry of the Tetracycline Antibiotics”, Medicinal Research 9, Dekker, New York, 1978; Noyee Development Corporation, “Tetracycline Manufacturing Processes” Chemical Process Reviews, Park Ridge, N.J., 2 volumes, 1969; R. C. Evans, “The Technology of the Tetracyclines”, Biochemical Reference Series 1, Quadrangle Press, New York, 1968; and H. F. Dowling, “Tetracycline”, Antibiotic Monographs, no. 3, Medical Encyclopedia, New York, 1955. Preferred Tc analogues for high level stimulation of transcription are anhydrotetracycline and doxycycline. A Tc analogue can be chosen which has reduced antibiotic activity compared to Tc. Examples of such Tc analogues are anhydrotetracycline and epioxytetracycline.

To induce shRNA expression in a cell in vitro, the cell is contacted with Tc or a Tc analogue by culturing the cell in a medium containing the compound. When culturing cells in vitro in the presence of Tc or Tc analogue, a preferred concentration range for the inducing agent is between about 10 and about 1000 ng/ml. Tc or a Tc analogue can be directly added to media in which cells are already being cultured, or more preferably for high levels of shRNA expression, cells are harvested from Tc-free media and cultured in fresh media containing Tc, or an analogue thereof.

To induce shRNA expression in vivo, cells within in a subject are contacted with Tc or a Tc analogue by administering the compound to the subject. The term “subject” is intended to include humans and other non-human mammals including monkeys, cows, goats, sheep, dogs, cats, rabbits, rats, mice, and transgenic and homologous recombinant species thereof. Furthermore, the term “subject” is intended to include plants, such as transgenic plants. When the inducing agent is administered to a human or animal subject, the dosage is adjusted to preferably achieve a serum concentration between about 0.05 and 1.0 μg/ml. Tc or a Tc analogue can be administered to a subject by any means effective for achieving an in vivo concentration sufficient for shRNA induction. Examples of suitable modes of administration include oral administration (e.g., dissolving the inducing agent in the drinking water), slow release pellets and implantation of a diffusion pump. To administer Tc or a Tc analogue to a transgenic plant, the inducing agent can be dissolved in water administered to the plant.

The ability to use different Tc analogues as inducing agents in this system allows for modulate the level of expression of a tet operator-linked nucleotide sequence. As demonstrated in Example 2, anhydrotetracycline and doxycycline have been found to be strong inducing agents. The increase in transcription of the target sequence is typically as high as 1000- to 2000-fold, and induction factors as high as 20,000 fold can be achieved. Tetracycline, chlorotetracycline and oxytetracycline have been found to be weaker inducing agents, i.e., the increase in transcription of a target sequence is in the range of about 10-fold. Thus, an appropriate tetracycline analogue is chosen as an inducing agent based upon the desired level of induction of shRNA expression. It is also possible to change the level of shRNA expression in a host cell or animal over time by changing the Tc analogue used as the inducing agent. For example, there may be situations where it is desirable to have a strong burst of shRNA expression initially and then have a sustained lower level of shRNA expression. Accordingly, an analogue which stimulates a high levels of transcription can be used initially as the inducing agent and then the inducing agent can be switched to an analogue which stimulates a lower level of transcription. Moreover, when regulating the expression of multiple nucleotide sequences (e.g., when one sequence is regulated by a one of class tet operator sequence(s) and the other is regulated by another class of tet operator sequence(s), as described above in Section III, Part C, above), it may be possible to independently vary the level of expression of each sequence depending upon which transcriptional activator fusion protein is used to regulate transcription and which Tc analogue(s) is used as the inducing agent. Different transcriptional activator fusion proteins are likely to exhibit different levels of responsiveness to Tc analogues. The level of induction of shRNA expression by a particular combination of transcriptional activator fusion protein and inducing agent (Tc or Tc analogue) can be determined by techniques described herein, (e.g., see Example 2). Additionally, the level of shRNA expression can be modulated by varying the concentration of the inducing agent. Thus, the expression system of the invention provides a mechanism not only for turning shRNA expression on or off, but also for “fine tuning” the level of shRNA expression at intermediate levels depending upon the type and concentration of inducing agent used.

IV. Constructs/Transgene

A construct is a recombinant nucleic acid, generally recombinant DNA, generated for the purpose of the expression of a specific nucleotide sequence(s), or is to be used in the construction of other recombinant nucleotide sequences. A transgene is a construct that has been or is designed to be incorporated into a cell, particularly a mammalian cell, that in turn becomes or is incorporated into a living animal such that the construct containing the nucleotide sequence is expressed (i.e., the mammalian cell is transformed with the transgene). The transgene may include a sequence (e.g., a shRNA-encoding sequence) that is endogenous or exogenous to the transgenic animal. A transgene may be present as an extrachromosomal element in some or all of the cells of a transgenic animal or, preferably, stably integrated into some or all of the cells, more preferably into the germline DNA of the animal (i.e., such that the transgene is transmitted to all or some of the animal's progeny), thereby directing expression of the product of the transgene in one or more cell types or tissues of the transgenic animal. Unless otherwise indicated, it will be assumed that a transgenic animal comprises stable changes to the chromosomes of germline cells. In a preferred embodiment, the transgene is present in the genome at a site such that it does not interfere with gene expression.

Such transgenic animals are created by introducing a transgenic construct of the invention into its genome using methods and vectors as described herein.

A transgenic construct of the invention includes the encoding sequence operably linked to an appropriate promoter sequence. The transgene optionally includes enhancer sequences and other non-coding sequences (for example, intron and/or 5′ or 3′ untranslated sequences).

V. Vectors and Host Cells

Another aspect of the invention pertains to vectors, preferably expression vectors, containing a construct of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

In general, expression vectors of utility in recombinant DNA techniques are often in the form of plasmids. Accordingly, in one embodiment, an expression vector of the invention is a plasmid. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Thus, in one embodiment, an expression vector of the invention is a viral-based vector. For example, replication defective retroviruses, adenoviruses and adeno-associated viruses can be used. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. The genome of adenovirus can be manipulated such that it encodes and expresses a regulatable shRNA construct, as described herein, but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Alternatively, an adeno-associated virus vector such as that described in Tratschin et al. (1985) Mol. Cell. Biol. 5:3251-3260 can be used to express a transactivator fusion protein. In a particular embodiment of the invention, an expression vector is not a viral vector.

The vectors of the invention comprise a shRNA-encoding nucleic acid operatively linked to one or more regulatory sequences (e.g., promoter sequences, e.g., Pol II or Pol III promoter sequences). The phrase “operably linked” is intended to mean that the nucleotide sequence of interest (e.g., the shRNA-encoding sequence) is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements, such as transcription termination signals (e.g., polyadenylation signals). Such regulatory sequences are described, for example, in Goeddel; Gene Expression Technology. Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). Other elements included in the design of a particular expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The vectors described herein can be introduced into cells or tissues by any one of a variety of known methods within the art. Such methods are described for example in Sambrook et al., Molecular Cloning: A Laboratory Manual, Cold Spring Harbor Laboratory, New York (1992), which is hereby incorporated by reference. See, also, Ausubel et al., Current Protocols in Molecular Biology, John Wiley and Sons, Baltimore, Md.(1989); Hitt et al., “Construction and propagation of human adenovirus vectors,” in Cell Biology: A Laboratory Handbook, Ed. J. E. Celis., Academic Press. 2.sup.nd Edition, Volume 1, pp: 500-512, 1998; Hitt et al., “Techniques for human adenovirus vector construction and characterization,” in Methods in Molecular Genetics, Ed. K. W. Adolph, Academic Press, Orlando, Fla., Volume 7B, pp:12-30, 1995; Hitt, et al., “Construction and propagation of human adenovirus vectors,” in Cell Biology: A Laboratory Handbook,” Ed. J. E. Celis. Academic Press. pp:479-490, 1994, also hereby incorporated by reference. The methods include, for example, stable or transient transfection, lipofection, electroporation and infection with recombinant viral vectors. The term “transfecting” or “transfection” is intended to encompass all conventional techniques for introducing nucleic acid into host cells, including calcium phosphate co-precipitation, DEAE-dextran-mediated transfection, lipofection, electroporation and microinjection. Suitable methods for transfecting host cells can be found in Sambrook et al. (Molecular Cloning: A Laboratory Manual, 2nd Edition, Cold Spring Harbor Laboratory press (1989)), and other laboratory textbooks.

The number of host cells transformed with a nucleic acid of the invention will depend, at least in part, upon the type of recombinant expression vector used and the type of transfection technique used. Nucleic acid can be introduced into a host cell transiently, or more typically, for long term regulation of gene expression, the nucleic acid is stably integrated into the genome of the host cell or remains as a stable episome in the host cell. Plasmid vectors introduced into mammalian cells are typically integrated into host cell DNA at only a low frequency. In order to identify these integrants, a gene that contains a selectable marker (e.g., drug resistance) is generally introduced into the host cells along with the nucleic acid of interest. Preferred selectable markers include those which confer resistance to certain drugs, such as G418 and hygromycin. Selectable markers can be introduced on a separate plasmid from the nucleic acid of interest or, are introduced on the same plasmid. Host cells transfected with a nucleic acid of the invention (e.g., a recombinant expression vector) and a gene for a selectable marker can be identified by selecting for cells using the selectable marker. For example, if the selectable marker encodes a gene conferring neomycin resistance, host cells which have taken up nucleic acid can be selected with G418. Cells that have incorporated the selectable marker gene will survive, while the other cells die.

Nucleic acid encoding a regulatable shRNA of the invention can be introduced into eukaryotic cells growing in culture in vitro by conventional transfection techniques (e.g., calcium phosphate precipitation, DEAE-dextran transfection, electroporation etc.). Nucleic acid can also be transferred into cells in vivo, for example by application of a delivery mechanism suitable for introduction of nucleic acid into cells in vivo, such as retroviral vectors (see e.g., Ferry, N et al. (1991) Proc. Natl. Acad. Sci. USA 88:8377-8381; and Kay, M. A. et al. (1992) Human Gene Therapy 3:641-647), adenoviral vectors (see e.g., Rosenfeld, M. A. (1992) Cell 68:143-155; and Herz, J. and Gerard, R. D. (1993) Proc. Natl. Acad. Sci. USA 90:2812-2816), receptor-mediated DNA uptake (see e.g., Wu, G. and Wu, C. H. (1988) J. Biol. Chem. 263:14621; Wilson et al. (1992) J. Biol. Chem. 267:963-967; and U.S. Pat. No. 5,166,320), direct injection of DNA (see e.g., Acsadi et al. (1991) Nature 332: 815-818; and Wolff et al. (1990) Science 247:1465-1468) or particle bombardment (see e.g., Cheng, L. et al. (1993) Proc. Natl. Acad. Sci. USA 90:4455-4459; and Zelenin, A. V. et al. (1993) FEBS Letters 315:29-32). Thus, for gene therapy purposes, cells can be modified in vitro and administered to a subject or, alternatively, cells can be directly modified in vivo.

Another aspect of the invention pertains to host cells into which a host construct of the invention has been introduced, i.e., a “recombinant host cell.” It is understood that the term “recombinant host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell, although eukaryotic cells are preferred. Exemplary eukaryotic cells include mammalian cells (such as Chinese hamster ovary cells (CHO) or COS cells). Other suitable host cells are known to those skilled in the art.

The host cells of the invention can also be used to produce nonhuman transgenic animals. The nonhuman transgenic animals can be used in screening assays designed to identify agents or compounds, e.g., drugs, pharmaceuticals, etc., which are capable of ameliorating detrimental symptoms of selected disorders, such as disease and disorders associated with mutant or aberrant gene expression, gain-of-function mutants and neurological diseases and disorders.

The present invention is also not limited to the use of the cell types and cell lines used herein. Cells from different tissues or different species (human, mouse, etc.) are also useful in the present invention.

VI. Construction of Transgenic Animals

In one aspect, the present invention provides a non-human animal whose genome contains a shRNA-encoding construct or transgene of the invention. The present invention further provides methods for making a transgenic non-human animal whose genome contains a shRNA-encoding construct or transgene of the invention.

The transgenic animal used in the methods of the invention can be, e.g., a mammal, a bird, a reptile or an amphibian. Suitable mammals for uses described herein include: rodents; ruminants; ungulates; domesticated mammals; and dairy animals. Preferred animals include: rodents, goats, sheep, camels, cows, pigs, horses, oxen, llamas, chickens, geese, and turkeys. In a preferred embodiment, the non-human animal is a mouse.

Various methods of making transgenic animals are known in the art (see, e.g., Watson, J. D., et al., “The Introduction of Foreign Genes Into Mice,” in Recombinant DNA, 2d Ed., W. H. Freeman & Co., New York (1992), pp. 255-272; Gordon, J. W., Intl. Rev. Cytol. 115:171-229 (1989); Jaenisch, R., Science 240: 1468-1474 (1989); Rossant, J., Neuron 2: 323-334 (1990)). An exemplary protocol for the production of a transgenic pig can be found in White and Yannoutsos, Current Topics in Complement Research: 64th Forum in Immunology, pp. 88-94; U.S. Pat. No. 5,523,226; U.S. Pat. No. 5,573,933; PCT Application WO93/25071; and PCT Application WO95/04744. An exemplary protocol for the production of a transgenic rat can be found in Bader and Ganten, Clinical and Experimental Pharmacology and Physiology, Supp. 3:S81-S87, 1996. An exemplary protocol for the production of a transgenic cow can be found in Transgenic Animal Technology, A Handbook, 1994, ed., Carl A. Pinkert, Academic Press, Inc. An exemplary protocol for the production of a transgenic sheep can be found in Transgenic Animal Technology, A Handbook, 1994, ed., Carl A. Pinkert, Academic Press, Inc. Several exemplary methods are set forth in more detail below.

A. Injection into the Pronucleus

Transgenic animals can be produced by introducing a nucleic acid construct according to the present invention into egg cells. The resulting egg cells are implanted into the uterus of a female for normal fetal development, and animals which develop and which carry the transgene are then backcrossed to create heterozygotes for the transgene. Embryonal target cells at various developmental stages are used to introduce the transgenes of the invention. Different methods are used depending on the stage of development of the embryonal target cell(s). Exemplary methods for introducing transgenes include, but are not limited to, microinjection of fertilized ovum or zygotes (Brinster, et al., Proc. Natl. Acad. Sci.USA (1985) 82: 4438-4442), and viral integration (Jaenisch R., Proc. Natl. Acad. Sci. USA (1976) 73: 1260-1264; Jahner, et al., Proc. Natl. Acad. Sci.USA (1985) 82: 6927-6931; Van der Putten, et al., (1985) Proc. Natl. Acad. Sci. (USA) 82: 6148-6152). Procedures for embryo manipulation and microinjection are described in, for example, Manipulating the Mouse Embryo (Cold Spring Harbor Laboratory Press, Cold Spring Harbor, N.Y., 1986, the contents of which are incorporated herein by reference). Similar methods are used for production of other transgenic animals.

In an exemplary embodiment, production of transgenic mice employs the following steps. Male and female mice, from a defined inbred genetic background, are mated. The mated female mice are previously treated with pregnant mare serum, PMS, to induce follicular growth and human chorionic gonadotropin, hCG, to induce ovulation. Following mating, the female is sacrificed and the fertilized eggs are removed from her uterine tubes. At this time, the pronuclei have not yet fused and it is possible to visualize them using light microscopy. In an alternative protocol, embryos can be harvested at varying developmental stages, e.g. blastocysts can be harvested. Embryos are recovered in a Dulbecco's modified phosphate buffered saline (DPBS) and maintained in Dulbecco's modified essential medium (DMEM) supplemented with 10% fetal bovine serum.

Foreign DNA or the recombinant construct (e.g. shRNA-encoding construct or transgene) is then microinjected (100-1000 molecules per egg) into a pronucleus. Microinjection of an expression construct can be performed using standard micro manipulators attached to a microscope. For instance, embryos are typically held in 100 microliter drops of DPBS under oil while being microinjected. DNA solution is microinjected into the male pronucleus. Successful injection is monitored by swelling of the pronucleus. Shortly thereafter, fusion of the pronuclei (a female pronucleus and a male pronucleus) occurs and, in some cases, foreign DNA inserts into (usually) one chromosome of the fertilized egg or zygote. Recombinant ES cells, which are prepared as set forth below, can be injected into blastocysts using similar techniques.

B. Embryonic Stem Cells

In another method of making transgenic mice, recombinant DNA molecules (e.g., constructs or transgenes) of the invention can be introduced into mouse embryonic stem (ES) cells. Resulting recombinant ES cells are then microinjected into mouse blastocysts using techniques similar to those set forth in the previous subsection.

ES cells are obtained from pre-implantation embryos and cultured in vitro (Evans, M J., et al., Nature 292: 154156 (1981); Bradley, M. O. et al., Nature 309: 255-258 (1984); Gossler, et al., Proc. Natl. Acad. Sci. (USA) 83:9065-9069 (1986); Robertson et al., Nature 322: 445448 (1986)). Any ES cell line that is capable of integrating into and becoming part of the germ line of a developing embryo, so as to create germ line transmission of the targeting construct, is suitable for use herein. For example, a mouse strain that can be used for production of ES cells is the 129J strain. A preferred ES cell line is murine cell line D3 (American Type Culture Collection catalog no. CRL 1934). The ES cells can be cultured and prepared for DNA insertion using methods known in the art and described in Robertson, Teratocarcinomas and Embryonic Stem Cells. A Practical Approach, E. J. Robertson, ed. IRL Press, Washington, D.C., 1987, in Bradley et al., Current Topics in Devel. Biol., 20:357-371, 1986 and in Hogan et al., Manipulating the Mouse Embryo: A Laboratory Manual, Cold Spring Harbor Laboratory Press, Cold Spring Harbor, N.Y., 1986, the contents of which are incorporated herein by reference.

The expression construct can be introduced into the ES cells by methods known in the art, e.g., those described in Sambrook et al., Molecular Cloning. A Laboratory Manual, 2^{nd} Ed., ed., Cold Spring Harbor laboratory Press: 1989, the contents of which are incorporated herein by reference. Suitable methods include, but are not limited to, electroporation, microinjection, and calcium phosphate treatment methods. The foreign DNA (e.g. construct or transgene) to be introduced into the ES cell is preferably linear.

After introduction of the expression construct, the ES cells are screened for the presence of the construct. The cells can be screened using a variety of methods. ES cell genomic DNA can be examined directly. For example, the DNA can be extracted from the ES cells using standard methods and the DNA can then be probed on a Southern blot with a probe or probes designed to hybridize specifically to the transgene. The genomic DNA can also be amplified by PCR with probes specifically designed to amplify DNA fragments of a particular size and sequence of the construct or transgene such that, only those cells containing the construct or transgene will generate DNA fragments of the proper size. Where a marker gene is employed in the construct, the cells of the animal can be tested for the presence of the marker gene. For example, where the marker gene is an antibiotic resistance gene, the cells can be cultured in the presence of an otherwise lethal concentration of antibiotic (e.g. G418 to select for neo). Those cells that survive have presumably integrated the transgene construct. If the marker gene is a gene that encodes an enzyme whose activity can be detected (e.g., β-galactosidase), the enzyme substrate can be added to the cells under suitable conditions, and the enzymatic activity can be analyzed.

C. Implantation

The zygote harboring a recombinant nucleic acid molecule of the invention (e.g. construct or transgene) is implanted into a pseudo-pregnant female mouse that was obtained by previous mating with a vasectomized male. In a general protocol, recipient females are anesthetized, paralumbar incisions are made to expose the oviducts, and the embryos are transformed into the ampullary region of the oviducts. The body wall is sutured and the skin closed with wound clips. The embryo develops for the full gestation period, and the surrogate mother delivers the potentially transgenic mice. Finally, the newborn mice are tested for the presence of the foreign or recombinant DNA. Of the eggs injected, on average 10% develop properly and produce mice. Of the mice born, on average one in four (25%) are transgenic for an overall efficiency of 2.5%. Once these mice are bred they transmit the foreign gene in a normal (Mendelian) fashion linked to a mouse chromosome.

D. Screening for the Presence of the Transgenic Construct

Transgenic animals can be identified after birth by standard protocols. DNA from tail tissue can be screened for the presence of the transgene construct, e.g., using southern blots and/or PCR. Offspring that appear to be mosaics are then crossed to each other if they are believed to carry the transgene in order to generate homozygous animals. If it is unclear whether the offspring will have germ line transmission, they can be crossed with a parental or other strain and the offspring screened for heterozygosity. The heterozygotes are identified by southern blots and/or PCR amplification of the DNA. The heterozygotes can then be crossed with each other to generate homozygous transgenic offspring. Homozygotes may be identified by southern blotting of equivalent amounts of genomic DNA from mice that are the product of this cross, as well as mice that are known heterozygotes and wild type mice. Probes to screen the southern blots can be designed based on the sequence of the construct or transgene, or a marker gene, or both.

Other means of identifying and characterizing the transgenic offspring are known in the art. For example, western blots can be used to assess the level of expression of a gene targeted for interference by probing with an antibody against the protein encoded by the target gene. Alternatively, an antibody against a marker gene product can be used, when a marker gene is expressed.

E. Mice Containing Multiple Transgenes

Transgenic mice expressing shRNAs as described herein can be crossed with mice that harbor additional transgene(s). Mice that are heterozygous or homozygous for shRNA expression can be generated and maintained using standard crossbreeding procedures. A preferred aspect of the invention features crossing mice that express a shRNA construct or transgene regulatable by a recombinase with mice expressing a corresponding recombinase. In a preferred embodiment, mice that express inducible silencing constructs or inducible desilencing constructs, as described herein, are crossed with mice expressing Cre. Such Cre expressing mice are known in the art and are publicly available, for example, from the Jackson Laboratory (Bar Harbor, Maine) or from Taconic.

The invention further pertains to cells derived from transgenic animals. Because certain modifications may occur in succeeding generations due to either mutation or environmental influences, such progeny may not, in fact, be identical to the parent cell, but are still included within the scope of the term as used herein.

VII. Uses and Methods of the Invention

The methods of the present invention will find great commercial application, for example in biotechnology, drug development and medicine. For example, in biotechnology, the ability to rapidly develop large numbers of transgenic animals with desired modulation of specific genes will allow for the analysis of gene function and the evaluation of compounds that potentially modulate gene expression, protein function, and are useful in treating a disease or disorder. In particular, by observing the effect of down-regulating specific genes in transgenic animals, the biological function of those genes may be determined, disease models may be established and drug targets may be validated. In medicine the methods of the invention may be used to treat patients suffering from particular diseases or disorders, for example, neurological diseases or disorders, or to confer immunity or resistance to particular pathogens. For example, specific cells may be infected in vivo or ex vivo with recombinant retrovirus encoding a siRNA that down-regulates the activity of a gene whose activity is associated with a particular disease or disorder.

A. Screening Assays

Cells and/or animals of the present invention may also be suitable for use in methods to identify and/or characterize potential pharmacological agents, e.g. identifying new pharmacological agents from a collection of test substances and/or characterizing mechanisms of action and/or side effects of known pharmacological agents.

Thus, the present invention also relates to a system for identifying and/or characterizing pharmacological agents comprising: (a) a cell (e.g., a eukaryotic cell) or organism (e.g., a eukaryotic non-human organism) containing a construct or transgene of the invention and (b) a test substance or a collection of test substances wherein pharmacological properties of said test substance or said collection are to be identified and/or characterized. Optionally, the system as described above can further comprise suitable controls.

Test compounds can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.)).

In a preferred embodiment, the library is a natural product library, e.g., a library produced by a bacterial, fungal, or yeast culture. In another preferred embodiment, the library is a synthetic compound library.

Compounds or agents identified according to such screening assays can be used therapeutically or prophylactically either alone or in combination, for example, with an shRNA of the invention, as described herein.

B. Knockout and/or Knockdown Cells or Organisms

A shRNAs (either known or identified by the methodologies of the present invention) can be used in a functional analysis of the corresponding target RNA (either known or identified by the methodologies of the present invention). Such a functional analysis is typically carried out in eukaryotic cells, or eukaryotic non-human organisms, preferably mammalian cells or organisms and most preferably human cells, e.g. cell lines such as HeLa or 293 or rodents, e.g. rats and mice. By administering a suitable shRNA molecule, a specific knockout or knockdown phenotype can be obtained in a target cell, e.g. in cell culture or in a target organism.

Thus, further subject matter of the invention includes cells (e.g., eukaryotic cells) or organisms (e.g., eukaryotic non-human organisms) exhibiting a target gene-specific knockout or knockdown phenotype resulting from a fully or at least partially deficient expression of at least one endogeneous target gene wherein said cell or organism is transfected with or administered, respectively, at least one shRNA, vector comprising DNA encoding said shRNA (or an shRNA precursor) capable of inhibiting the expression of the target gene. It should be noted that the present invention allows a target-specific knockout or knockdown of several different endogeneous genes based on the specificity of the shRNA(s) transfected or administered.

Gene-specific knockout or knockdown phenotypes of cells or non-human organisms, particularly of human cells or non-human mammals may be used in analytic procedures, e.g. in the functional and/or phenotypical analysis of complex physiological processes such as analysis of gene expression profiles and/or proteomes. Preferably the analysis is carried out by high throughput methods using oligonucleotide based chips.

Using RNAi based knockout or knockdown technologies, the expression of an endogeneous target gene may be inhibited in a target cell or a target organism. The endogeneous gene may be complemented by an exogenous target nucleic acid coding for the target protein or a variant or mutated form of the target protein, e.g. a gene or a DNA, which may optionally be fused to a further nucleic acid sequence encoding a detectable peptide or polypeptide, e.g. an affinity tag, particularly a multiple affinity tag.

Variants or mutated forms of the target gene differ from the endogeneous target gene in that they encode a gene product which differs from the endogeneous gene product on the amino acid level by substitutions, insertions and/or deletions of single or multiple amino acids. The variants or mutated forms may have the same biological activity as the endogeneous target gene. On the other hand, the variant or mutated target gene may also have a biological activity, which differs from the biological activity of the endogeneous target gene, e.g. a partially deleted activity, a completely deleted activity, an enhanced activity etc. The complementation may be accomplished by compressing the polypeptide encoded by the endogeneous nucleic acid, e.g. a fusion protein comprising the target protein and the affinity tag and the double stranded RNA molecule for knocking out the endogeneous gene in the target cell. This compression may be accomplished by using a suitable expression vector expressing both the polypeptide encoded by the endogenous nucleic acid, e.g. the tag-modified target protein and the double stranded RNA molecule or alternatively by using a combination of expression vectors. Proteins and protein complexes which are synthesized de novo in the target cell will contain the exogenous gene product, e.g., the modified fusion protein. In order to avoid suppression of the exogenous gene product by the siRNA molecule, the nicleotide sequence encoding the exogenous nucleic acid may be altered at the DNA level (with or without causing mutations on the amino acid level) in the part of the sequence which: so is homologous to the siRNA molecule. Alternatively, the endogeneous target gene may be complemented by corresponding nucleotide sequences from other species, e.g. from mouse.

C. Functional Genomics and/or Proteomics

Preferred applications for the cell or organism of the invention include the analysis of gene expression profiles and/or proteomes. In an especially preferred embodiment an analysis of a variant or mutant form of one or several target proteins is carried out, wherein said variant or mutant forms are reintroduced into the cell or organism by an exogenous target nucleic acid as described above. The combination of knockout of an endogeneous gene and rescue by using mutated, e.g. partially deleted exogenous target has advantages compared to the use of a knockout cell. Further, this method is particularly suitable for identifying functional domains of the targeted protein. In a further preferred embodiment a comparison, e.g. of gene expression profiles and/or proteomes and/or phenotypic characteristics of at least two cells or organisms is carried out. These organisms are selected from: (i) a control cell or control organism without target gene inhibition, (ii) a cell or organism with target gene inhibition and (iii) a cell or organism with target gene inhibition plus target gene complementation by an exogenous target nucleic acid.

Furthermore, the RNA knockout complementation method may be used for its preparative purposes, e.g. for the affinity purification of proteins or protein complexes from eukaryotic cells, particularly mammalian cells and more particularly human cells. In this embodiment of the invention, the exogenous target nucleic acid preferably codes for a target protein which is fused to art affinity tag. This method is suitable for functional proteome analysis in mammalian cells, particularly human cells. Another utility of the present invention could be a method of identifying gene function in an organism comprising the use of shRNA to inhibit the activity of a target gene of previously unknown function. Instead of the time consuming and laborious isolation of mutants by traditional genetic screening, functional genomics would envision determining the function of uncharacterized genes by employing the invention to reduce the amount and/or alter the timing of target gene activity. The invention could be used in determining potential targets for pharmaceutics, understanding normal and pathological events associated with development, determining signaling pathways responsible for postnatal development/aging, and the like.

The ease with which RNA can be introduced into an intact cell/organism containing the target gene allows the present invention to be used in high throughput screening (HTS). Solutions containing shRNAs that are capable of inhibiting the different expressed genes can be placed into individual wells positioned on a microtiter plate as an ordered array, and intact cells/organisms in each well can be assayed for any changes or modifications in behavior or development due to inhibition of target gene activity. The amplified RNA can be fed directly to, injected into, the cell/organism containing the target gene. Alternatively, the shRNA can be produced from a vector, as described herein. Vectors can be injected into, the cell/organism containing the target gene. The function of the target gene can be assayed from the effects it has on the cell/organism when gene activity is inhibited. This screening could be amenable to small subjects that can be processed in large number, for example: arabidopsis, bacteria, drosophila, fungi, nematodes, viruses, zebrafish, and tissue culture cells derived from mammals. A nematode or other organism that produces a colorimetric, fluorogenic, or luminescent signal in response to a regulated promoter (e.g., transfected with a reporter gene construct) can be assayed in an HTS format.

The HTS approach may identify new drug targets. The potential drug targets may also be validated using the present invention. For example, a particular disease phenotype might be induced by a gene mutation or a chemical. RNAi may be used to down-regulate genes and some of these down-regulations might lead to the reversal of the disease phenotype. These genes are potential drug targets. Compounds may be identified to inhibit these genes to treat the disease phenotype.

D. Viral Delivery Vehicles

One challenge that must be met to realize therapeutic applications of RNAi technologies is the development of systems to deliver siRNAs efficiently into mammalian cells. Towards that end, plasmids have been designed expressing short hairpin RNAs, or stem-loop RNA structures, driven by RNA polymerase III (pol III) promoters (T. R. Brummelkamp et al. Science (2002) 296:550-553; P. J. Paddison et al., Genes Dev. (2002) 16:948-958). The hairpin RNAs are processed to generate siRNAs in cells and thereby induce gene silencing. Pol III promoters are advantageous because their transcripts are not necessarily post-transcriptionally modified, and because they are highly active when introduced in mammalian cells. Polymerase II (pol II) promoters may offer advantages to pol III promoters, including being more easily incorporated into viral expression vectors, such as retroviral and adeno-associated viral vectors, and the existence of inducible and tissue specific pol II dependent promoters. In particular embodiments of the invention, the shRNA sequence is expressed from a Pol III promoter, e.g., U6 promoter. In other embodiments of the invention, the shRNA sequence is under the control of and expressed from a Pol II promoter, e.g., Ubiquitin C promoter.

The limitation of plasmid-based siRNA delivery systems is their dependence on cell transfection methods, which are rarely efficient and limited primarily to established cell lines. Viral based strategies would offer the significant advantage of allowing for efficient delivery to cell lines as well as primary cells.

E. Methods of Treatment

The present invention provides shRNA-expressing constructs that are useful clinically (e.g., in certain prophylactic and/or therapeutic applications). For example, shRNAs can be used, for example, as prophylactic and/or therapeutic agents in the treatment of diseases or disorders associated with unwanted or aberrant expression of the corresponding target gene.

In one embodiment, the invention provides for prophylactic methods of treating a subject at risk of (or susceptible to) a disease or disorder, for example, a disease or disorder associated with aberrant or unwanted target gene expression or activity. Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject. The appropriate agent can be determined based on screening assays described herein.

In another embodiment, the invention provides for therapeutic methods of treating a subject having a disease or disorder, for example, a disease or disorder associated with aberrant or unwanted target gene expression or activity. In an exemplary embodiment, the modulatory method of the invention involves contacting a cell capable of expressing target gene with a therapeutic agent that is specific for the target gene or protein (e.g., is specific for the mRNA encoded by said gene or specifying the amino acid sequence of said protein) such that expression or one or more of the activities of target protein is modulated. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

“Treatment”, or “treating” as used herein, is defined as the application or administration of a prophylactic or therapeutic agent to a patient, or application or administration of a prophylactic or therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

Knowledge of shRNAs and their targets would allow specific modulation of shRNA systems to treat any of a number of disorders (including cancer, inflammation, neuronal disorders, etc.). Manipulating shRNA regulation of translation of these genes is a novel, powerful, and specific method for treating these disorders.

VIII. Pharmacogenomics and Pharmaceutical Compositions

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

With regards to the above-described agents for prophylactic and/or therapeutic treatments, the agents are routinely incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

When administering shRNAs, it may be advantageous to chemically modify the shRNA in order to increase in vivo stability. Preferred modifications stabilize the shRNA against degradation by cellular nucleases.

The pharmaceutical compositions can be included in a container, pack, or dispenser together with instructions for administration.

This invention is further illustrated by the following examples which should not be construed as limiting. The contents of all references, patents and published patent applications cited throughout this application are incorporated herein by reference.

### EXAMPLES

Mutations in Cu, Zn superoxide dismutase (SOD1) gene cause a subset of amyotrophic lateral sclerosis, a neurodegenerative disease that leads to motor neuron degeneration, paralysis and death (Brown and Robberecht, 2001; Siddique and Lalani, 2002). It has been well established that mutant SOD1 causes motor neuron degeneration by acquisition of a toxic property (Cleveland and Rothstein, 2001). However, neither the molecular basis of this toxic property nor mechanism that leads to motor neuron death is understood. Because of this incomplete understanding of the disease mechanism, rational design of therapy has not produced robust efficacious outcomes. On the other hand, because the toxicity that kills motor neurons originates from the mutated protein (Cleveland and Rothstein, 2001), decrease of the mutant protein should alleviate or even prevent the disease. The following examples demonstrate that RNAi can be used to target mutant SOD1 protein without detrimentally affecting normal SOD1. In particular, the examples teach regulatable silencing of mutant SOD1 in transgenic mice. As described in detail above, the invention features constructs that provide for inducible shRNA expression (and, in turn, gene silencing) when appropriately introduced into cells or animals. These examples teach a shRNA capable of silencing mutant SOD1 as the target gene although the constructs work equally well for the silencing of any target gene, be it a mutant target gene or normal (wild type) target gene.

### Example 1

### Generation of Inducible Desilencing Constructs containing a Pol III Promoter

The first type of exemplary construct provided in the instant invention is an inducible desilencing construct that expresses shRNA against mutant SOD1, but upon exposure to Cre recombinase the transgene is excised, thereby inhibiting shRNA expression and its silencing of mutant SOD1 expression.

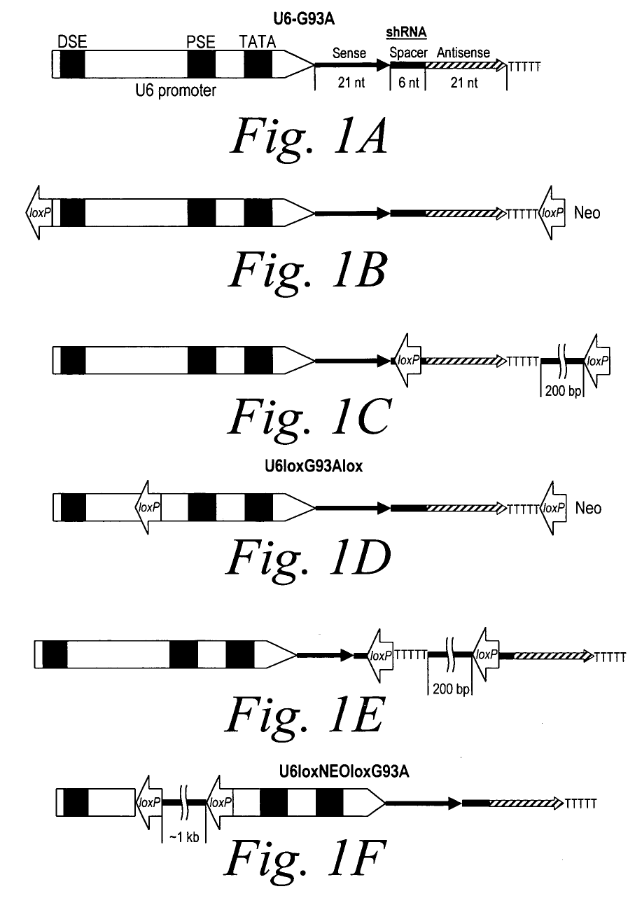

With respect to this first class of constructs, constructs B-D were generated as follows. Construct B contains two loxP sites flanking the entire U6-G93A expression unit (FIG. 1B). Therefore, the entire transgene is looped out after Cre recombination. Construct C, depicted in FIG. 1C, contains a loxP in the loop region, and another loxP in the 3′ end of the putative shG93A transcript. Because a minimum of 82-bp between the two loxP sites is required for Cre-mediated excision, approximately 200 base pair intervening sequence is inserted in front of the second loxP site to increase the length of the sequence between the two loxP sites so that this sequence can be excised efficiently. Cre-mediated recombination removes the antisense strand. The resulting construct will be not able to produce shRNA and cannot mediate RNAi, thus causing desilencing. Construct D (also called U6loxG93Alox), depicted in FIG. 1D and set forth in SEQ ID NO: 2, has one loxP inserted in the U6 promoter region and another loxP at the 3′ end of the shRNA. Results indicate that this insertion does not affect the function of the U6 promoter. Cre recombination removes a large part of the U6 promoter and the entire shRNA coding sequence, and the looped-out circle cannot produce significant transcription because it lacks an obligatory promoter element DSE (Bark et al., 1987; Carbon et al., 1987; Kunkel and Pederson, 1988). Construct D expresses shG93A has been constructed and verified by sequencing and by in vitro Cre-mediated recombination reactions.

### Example 2

### Generation of Inducible Silencing Constructs containing a Pol III Promoter

A second type of construct provided herein is an inducible silencing construct that cannot express shRNA, but upon exposure to Cre recombinase the transgene activates and expresses the shRNA, thereby silencing mutant SOD1 expression. This second class of constructs, which are normally inactive and do not express shRNA, and which, upon exposure to Cre recombinase, result in expression of the shRNA, are called inducible silencing constructs.

An example of this second class of constructs, construct E in FIG. 1, is a sequence containing a string of five Ts and a ˜200 bp spacer sequence inserted between the sense and antisense strand of the shRNA coding sequence. Flanking this insertion are loxP sites on both sides (FIG. 1E). The hairpin cannot be expressed because the transcription stops at the five Ts before it reaches the antisense strand. Cre recombinase removes these five Ts along with the spacer sequence, thereby allowing the synthesis of the entire hairpin.

Another example of this second class of constructs is Construct F (also called U6loxNEOloxG93A), depicted in FIG. 1 and the sequence of which is set forth in SEQ ID NO: 3. Construct F contains a U6 promoter that is disrupted by insertion of a non-specific piece of DNA (˜1 kb from NEO coding region), which is flanked by loxP sites on both sides (FIG. 1F) so that it can be excised upon exposure to Cre recombinase. The U6 promoter structure was examined for an insertion site that would interrupt the promoter function, but upon Cre-mediated recombination, the promoter function could recover (i.e., the promoter function is not affected by leaving one loxP sequence in it). The U6 promoter is composed of 250 nucleotides 5′ to the U6 snRNA (Paule et al., 2000). It contains 3 essential elements that are indispensable for the promoter function: the distal sequence element (DSE), the proximal sequence element (PSE) and the TATA box (see FIG. 1). Early work has found that the number of nucleotides is strictly defined between the TATA box and the transcription initiation site (24 nucleotides), and between the PSE and TATA box (17 nucleotides) (Lescure et al., 1991; Goomer and Kunkel, 1992). Alteration of the distances between these elements seriously impaired the promoter function. Because the length of a single loxP is 34 nucleotides, it is longer than the distance between the PSE and TATA, and between TATA and the transcription initiation site. This precludes these two sites for placing the disruption sequence. To avoid interfering with the promoter function after Cre-mediated recombination, a single loxP site was inserted into the segment between the DSE and PSE, at ˜60 nucleotide from the PSE (FIG. 1F). This particular region was chosen because it was not bound to chromatin and therefore, without being bound by theory, it was believed that an insertion of a new sequence would not interfere with nucleosome formation and affect promoter function.

### Example 3

### Analysis of Inducible Silencing Constructs

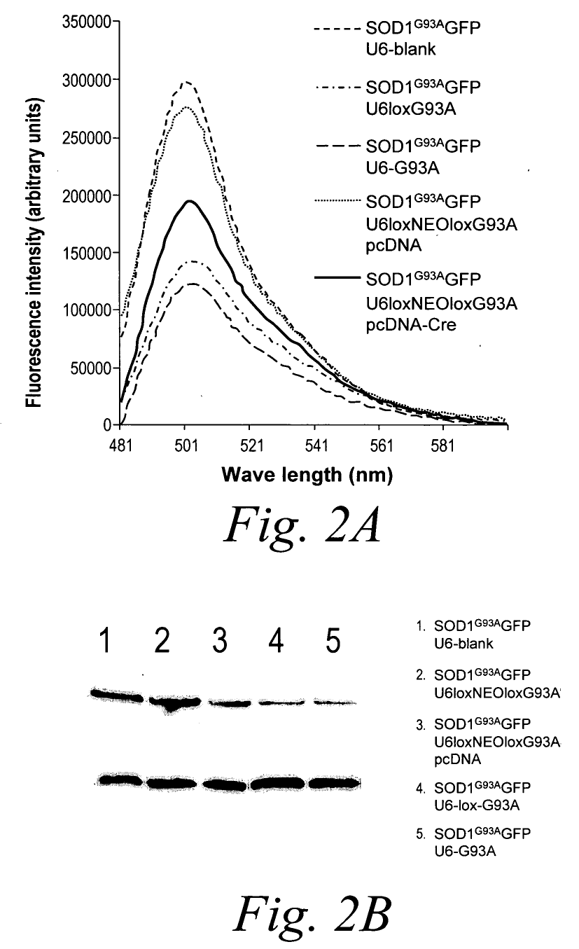

SOD1^{G93A}GFP expression was quantified in cell lysates by emission scanning using a fluorometer (n=3) according to the published protocol (Chiu et al. 2002) (FIG. 2A). Construct A in which only one loxP site was placed within the promoter region was tested by cotransfection with the SOD1^{G93A}GFP vector and was found to display a similar a silencing effect as the original U6-G93A construct (FIG. 2, compare lanes 4 and 5). These results indicated that insertion of one loxP site does not interrupt the normal function of the U6 promoter.

Subsequently, a ˜1 kb segment that was PCR-amplified from the Neomycin (NEO) coding region (without the promoter) and that contained loxP sequences flanking both sides of the Neo gene was inserted into the construct (named U6loxNEOloxG93A, see FIG. 1F). When this construct was tested, the construct did not silence SOD1^{G93A}GFP expression in the absence of Cre (FIG. 2), indicating shG93A expression was blocked and the U6 promoter function was disrupted. Cre expression led to partial silencing of SOD1^{G93A}GFP expression (FIG. 2). These results demonstrate that (1) the U6 promoter can tolerate insertion of one loxP site between the PSE and DSE, and (2) the U6loxNEOloxG93A shRNA-synthesizing construct functioned as designed.

FIG. 2B depicts detection of the transfected SOD1G93AGFP and the endogenous human SOD1 proteins in 293 cells by Western blot. Cells that were cotransfected with U6-blank or U6loxNEOloxG93A demonstrate high SOD^{G93A} levels (see lanes 1 and 2 of FIG. 2B). In cells contransfected with U6-G93A, U6loxG93A (a loxP site was inserted into the U6 promoter) or U6loxNEOloxG93A and pcDNA-Cre, the SOD^{G93A} levels were suppressed (see lanes 3-5 of FIG. 2B).

### Example 4

### Transgenic Animals

Both constructs (linearized), U6loxG93Alox and U6loxNEOloxG93A, were injected into fertilized mouse eggs at the University of Massachusetts Medical School transgenic core facility to produce transgenic mice. The transgenic mice were identified by PCR using PCR primers that selectively amplify the respective constructs. Mice so identified (founder mice) were then crossed with CMV-Cre mice (obtained from Jackson Laboratory). This line was created using a Cre construct under the control of human cytomegalovirus (CMV) minimal promoter and is well characterized (Schwenk et al., 1995). The transgene was incorporated into the X chromosome. Nevertheless, when this transgenic line was crossed with a loxPstoploxP-LacZ reporter transgenic line, all cells in the female doubly transgenic progenies expressed lacZ. This indicates that the stop sequence was successfully excised in all cells. This is apparently due to the early expression of Cre during embryonic development before the onset X-chromosome inactivation at the implantation stage (Schwenk et al., 1995). A possible alternative to this line is β-actin-Cre mice, which express Cre ubiquitously (Lewandoski et al., 1997). This alternative line is also available from Jackson Laboratory. After the crossing, various tissues from both doubly and singly transgenic mice are characterized, e.g., by determining tissue expression pattern of shRNA by Northern blot analysis, and for completeness of the transgene recombination by Southern blot analysis.

Lines that express high levels of shG93A in all tissues and execute Cre recombination in all tissues can further be crossed with SOD₁^{G93A} mice to make triply transgenic mice. The SOD1 expression pattern is examined using Western blots of various tissues, and semi-quantitative in situ hybridization and immunofluorescence on tissue sections. The extent of disease phenotype inhibition is also examined.

### Example 5

### Tetracycline Regulatable Silencing Constructs containing POL II Promoters

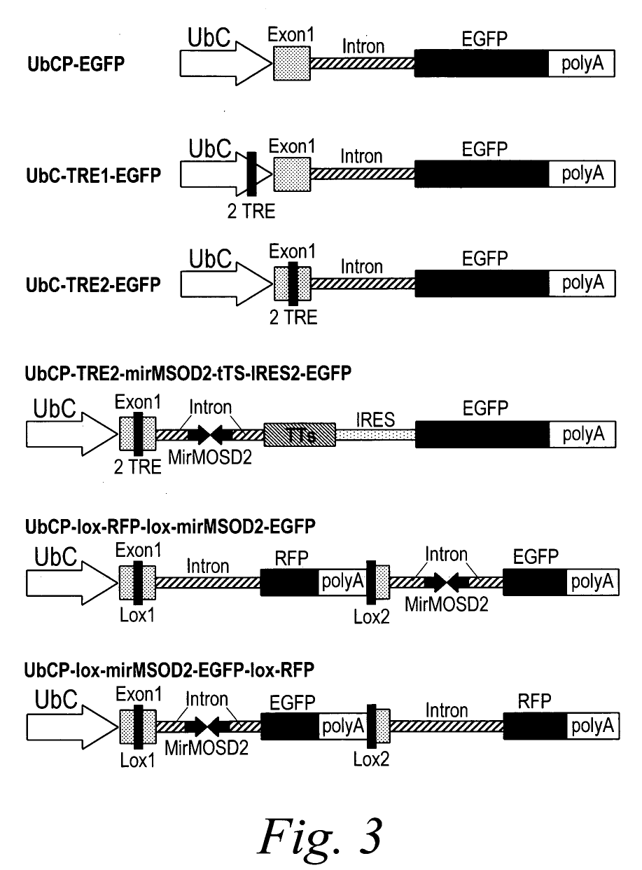

Ubiquitin C promoter (UbCP) is a Pol II promoter that is active ubiquitously and constitutively in all mammalian cells (Lois, 2002). Previous work has mapped this promoter and it has been used widely in culture and in animals to express genes (Li, 2003). As provided in this Example, the Ubiquitin C promoter has been modified so that it can be regulated temporally and spatially in culture and in animals. The basic promoter elements include a 335 bp 5′ promoter region, exon 1 and the first intron. Exon 1 is a non-coding exon but may contain some regulatory elements for ubiquitin C expression. The basic construct consists of the above promoter elements, cDNA coding for the EGFP protein (Invitrogen) and the SV40 poly A element (UbCP-EGFP in FIG. 3, SEQ ID NO: 4).

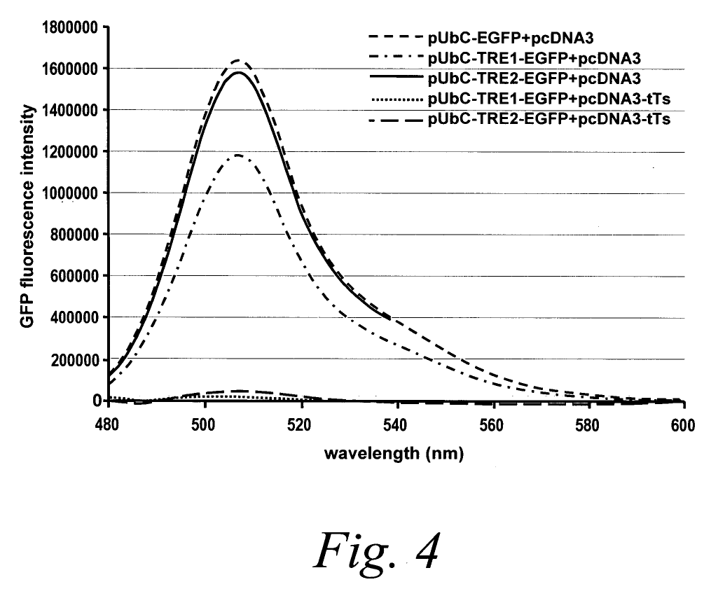

To create an expression vector that can be regulated by tetracycline, two constructs were designed. In the first construct, two tetracycline responsive elements (TREs) were placed in the UbCP (FIG. 3, UbCP-TRE1-EGFP; SEQ ID NO: 5). In the second construct, the two TREs were placed in the first exon (FIG. 3, UbCP-TRE2-EGFP; SEQ ID NO: 6). Both constructs expressed EGFP, although construct 2 (UbCP-TRE2-EGFP) provided better expression than construct 1, (UbCP-TRE1-EGFP). UbCP-TRE2-EGFP expressed at the same level as unmodified UbCP-EGFP, while UbCP-TRE1-EGFP expressed at lower levels (FIG. 4). Thus, UbCP-TRE2-EGFP offers advantages over UbCP-TRE1-EGFP. As expected, the expression of EGFP could be suppressed in the presence of tTS, a repressor that binds to TREs (FIG. 4).

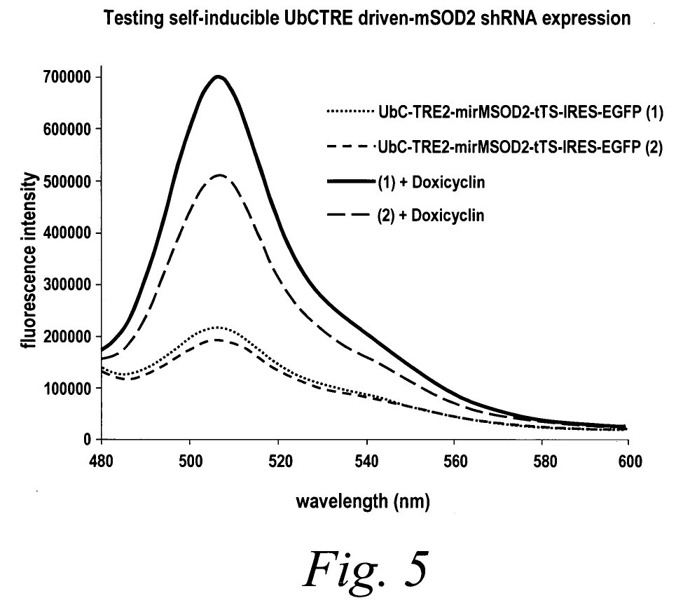

Next, to use this expression vector system to express shRNAs, the vector UbC-TRE2-mirMSOD2-tTS-IRES-EGFP (FIG. 3, SEQ ID NO: 7) was created. This is a self repressing construct, wherein UbCP directs synthesis of an mRNA that contains exon1, intron 1 containing a sequence for mirMSOD2 expression (see below), tTS coding sequence, IRES (internal ribosomal entry site) and EGFP coding sequence. The mirMSOD1 sequence directs expression of a shRNA targeting mouse SOD2 gene, although this can be a sequence coding for a short hairpin RNA targeting any other gene (see further description below). In the absence of the inducer doxicyclin (a tetracycline analogue), the UbCP directs synthesis of tTS and EGFP. However, this expression is suppressed by tTS because it binds to TRE and blocks the promoter. Upon addition of the inducer doxicyclin, which binds to tTS and prevents it from binding to the TRE, the suppression of transcription is released and transcription increases. The construct was tested by transfecting UbC-TRE2-mirMSOD2-tTS-IRES-EGFP into HEK293 cells. In two separate experiments, when vector was transfected in the absence of doxicyclin, the basal level of expression of EGFP was suppressed at low levels. Upon addition of doxicyclin, expression of EGFP was increased (FIG. 5).

In a different design, the tTS can alternatively be provided by an independent Pol II promoter. In that case, the background expression of the mirMSOD2 and EGFP are expected to be lower than what has been observed in UbC-TRE2-mirMSOD2-tTS-IRES-EGFP, as shown in FIG. 5.

### Example 6

### Cre/Lox Regulatable Silencing Constructs containing Pol II Promoters

Vectors were next designed to allow shRNA expression from a Pol II promoter to be controlled in specific cells. The vector UbCP-lox-RFP-lox-mirMSOD2-EGFP (FIG. 3; SEQ ID NO: 8) was created. In this construct, UbCP normally transcribes RFP mRNA, which terminates at the poly site. However, because the RPF gene is flanked by two loxP sites, upon exposure to cre, the RFP gene is excised and the construct is converted to UbCP-lox-mirMSOD2-EGFP. The resulting recombined sequence transcribes a message that contains an intron with an shRNA against the mouse SOD2 gene (or any other genes) and an EGFP gene.

A second, complementary vector UbCP-lox-mirMSOD2-EGFP-lox-RFP (FIG. 3; SEQ ID NO: 9) was also created. In this vector, the UbCP normally directs synthesis of the hairpin and EGFP. However, upon exposure to cre, the hairpin and EGFP are excised, thus preventing hairpin expression, while the RFP gene is under control of the UbCP, thus activating RFP expression.

To test these constructs, both UbCP-lox-RFP-lox-mirMSOD2-EGFP and UbCP-lox-mirMSOD2-EGFP-lox-RFP were cotransfected with either pcDNA3 empty vector or pcDNA3-cre. When UbCP-lox-RFP-lox-mirMSOD2-EGFP was cotransfected with pcDNA3 empty vector, RFP expression was observed by microscopic analysis. When this same vector was cotransfected with pcDNA3-cre, GFP was expressed. The converse was true for the UbCP-lox-mirMSOD2-EGFP-lox-RFP construct. When UbCP-lox-mirMSOD2-EGFP-lox-RFP was cotransfected with pcDNA3 empty vector, GFP was expressed. When this same construct was cotransfected with pcDNA3-cre, RFP was expressed. These data demonstrated that these constructs function according to their design.

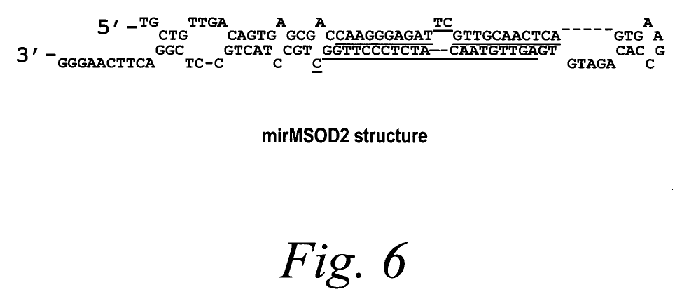

The mirMSOD2 is an shRNA that incorporates a miRNA hairpin structure (FIG. 6; SEQ ID NO: 1). MirMSOD2 is placed in intron 1 and is intended to silences mouse SOD2 (see FIG. 3). When intron 1 is spliced out, mirMSOD2 will be processed by Drosha to produce an shRNA, which in turn will be exported out of the nucleus by exportin 5. The shRNA will be further processed by Dicer in the cytoplasm, form a complex with RISC and mediate RNAi against the SOD2 mRNA. Thus, SOD2 expression should be inhibited when the intron 1 is expressed. The DNA sequence corresponding to mirMSOD2 is provided in SEQ ID NO: 1. It will be understood that a mirMSOD2 shRNA sequence is the corresponding RNA sequence in which the thymidines are replaced by uridines.

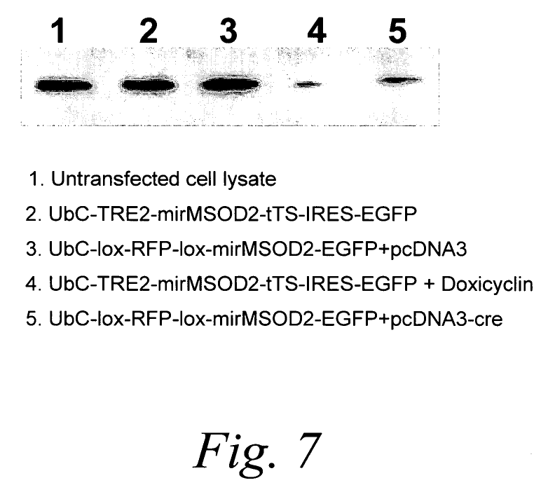

To test whether SOD2 expression was inhibited when intron 1 was expressed, mouse NF-1 cells were transfected with UbCP-TRE2-mirMSOD2-tTS-IRES-EGFP. Doxicyclin induced dramatic inhibition of expression of the endogenous SOD2 gene (FIG. 7, compare lane 2 with lane 4). Likewise, when UbCP-lox-RFP-lox-mirMSOD2-EGF was cotransfected with pcDNA3-cre, dramatic inhibition of SOD2 expression was observed (FIG. 7, compare lane 3 with lane 5). These results indicated that the mirMSOD2 hairpin vectors function as designed.

### Discussion of Examples 5 and 6

The regulatable shRNA expression system utilizing a Pol II promoter, as described in Examples 5 and 6, should function in vivo because (1) Pol II is the promoter used endogenously to synthesize miRNA, and (2) the endogenous miRNA structure has been applied in designing the shRNA. In addition, because a wide range of Pol II promoters with different temporal and spatial control in animals have already been characterized, the system provided herein can be used for inducing silencing at specific developmental stages or in specific cell types. Furthermore, the constructs of the instant invention solve the common problem when using RNAi in vivo of determining in which cell types the shRNA is expressed. Because the constructs provided herein containing a Pol II promoter direct expression of shRNA and marker proteins (e.g., GFP or RFP) at the same time, such marker protein expression marks the cells in which the shRNAs are expressed.

The following references are incorporated herein by reference:
Chen Y, Stamatoyannopoulos G, Song C-Z (2003) Down-Regulation of CXCR4 by Inducible Small Interfering RNA Inhibits Breast Cancer Cell Invasion in Vitro. Cancer Res 63:4801-4804.
Coumoul X, Li W, Wang R-H, Deng C (2004) Inducible suppression of Fgfr2 and Survivin in ES cells using a combination of the RNA interference (RNAi) and the Cre-LoxP system. Nucl Acids Res 32:e85-.
Gupta S, Schoer R A, Egan J E, Hannon G J, Mittal V (2004) From the Cover: Inducible, reversible, and stable RNA interference in mammalian cells. PNAS 101:1927-1932.
Kasim V, Miyagishi M, Taira K (2004) Control of siRNA expression using the Cre-loxP recombination system. Nucl Acids Res 32:e66-.
Li X, Makela S, Streng T, Santti R, Poutanen M (2003) Phenotype characteristics of transgenic male mice expressing human aromatase under ubiquitin C promoter. The Journal of Steroid Biochemistry and Molecular Biology 86:469-476.
Lois C, Hong E J, Pease S, Brown E J, Baltimore D (2002) Germline transmission and tissue-specific expression of transgenes delivered by lentiviral vectors. Science 295:868-872.
Matsukura S, Jones P A, Takai D (2003) Establishment of conditional vectors for hairpin siRNA knockdowns. Nucl Acids Res 31 :e77-.
Tiscornia G, Tergaonkar V, Galimi F, Verma I M (2004) From The Cover: CRE recombinase-inducible RNA interference mediated by lentiviral vectors. PNAS 101:7347-7351.
van de Wetering M, Oving I, Muncan V, Pon Fong M T, Brantjes H, van Leenen D, Holstege F C, Brummelkamp T R, Agami R, Clevers H (2003) Specific inhibition of gene expression using a stably integrated, inducible small-interfering-RNA vector. EMBO Rep 4:609-615.
Zeng Y, Cullen B R (2003) Sequence requirements for micro RNA processing and function in human cells. Rna 9:112-123.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A construct comprising a U6 promoter operably linked to a shRNA encoding nucleic acid sequence, the construct further comprising a first loxP site upstream of the promoter and a second loxP site downstream of the shRNA encoding sequence, the loxP sites being in the same orientation such that the promoter and encoding sequences are excisable upon exposure to Cre.

2. A construct comprising a U6 promoter operably linked to a shRNA encoding nucleic acid sequence, the shRNA encoding sequence comprising a first stem-encoding portion, a loop-encoding portion, and a second stem-encoding portion, wherein the construct further comprises spacer DNA downstream of the shRNA encoding sequence, a second loxP site downstream of the spacer DNA, and a first loxP site within the loop-encoding portion of the shRNA encoding sequence, the loxP sites being in the same orientation such that the spacer DNA and second stem-encoding sequence are excisable upon exposure to Cre.

3. A construct comprising a U6 promoter operably linked to a shRNA encoding nucleic acid sequence, the U6 promoter comprising (a) a distal sequence element (DSE); (b) a proximal sequence element (PSE); and (b) a TATA box, operably linked, wherein the construct further comprises a first loxP site downstream of the shRNA encoding sequence, and a second loxP site between the DSE and the PSE, the loxP sites being in the same orientation such that the shRNA encoding sequences and a portion of the promoter comprising the PSE and the TATA box are excisable upon exposure to Cre.

4. A construct comprising a U6 promoter operably linked to a shRNA encoding nucleic acid sequence, the shRNA encoding sequence comprising a first stem-encoding portion, a loop-encoding portion, and a second stem-encoding portion, the loop-encoding portion comprising a first loxP site operably linked to a transcription termination signal upstream of a spacer DNA and a second loxP site, the loxP sites being in the same orientation such that the first loxP site, termination signal and spacer DNA are excisable upon exposure to Cre.

5. A construct comprising a U6 promoter operably linked to a shRNA encoding nucleic acid sequence, the U6 promoter comprising (a) a distal sequence element (DSE); (b) a proximal sequence element (PSE); and (b) a TATA box, operably linked, wherein the construct further comprises a first loxP site and a second loxP site, said sites being interrupted by spacer DNA, between the DSE and the PSE, the loxP sites being in the same orientation such that a loxP site and the spacer DNA are excisable upon exposure to Cre.

6. An inducible desilencing construct for the expression of a shRNA, the construct comprising a promoter element operably linked to a shRNA encoding element and further comprising a first and second recombinase-sensitive element in an appropriate orientation such that all or a portion of the promoter or shRNA encoding element is excisable upon exposure to Cre.

7. The construct of claim 6, wherein the first recombinase-sensitive element is upstream of the promoter and the second recombinase-sensitive element is downstream of the shRNA encoding element.

8. The construct of claim 6, wherein the first recombinase-sensitive element is within the shRNA encoding element and the second recombinase-sensitive element is downstream of the shRNA encoding element.

9. The construct of claim 8, wherein the first element is within a loop portion of the shRNA encoding element.

10. The construct of claim 8, wherein the construct further includes a spacer nucleotide sequence between the shRNA encoding element and the second recombinase-sensitive element.

11. The construct of claim 10, wherein the spacer is between 50 and 200 nucleotides in length.

12. The construct of claim 6, wherein the first recombinase-sensitive element is within the promoter and the second recombinase-sensitive element is downstream of the shRNA encoding element.

13. The construct of claim 12, wherein the first recombinase-sensitive element is downstream of at least one obligatory element in said promoter.

14. The construct of claim 12, wherein the first recombinase element is downstream of a DSE element.

15. An inducible silencing construct for the expression of a shRNA, the construct comprising a promoter element operably linked to a shRNA encoding element, the promoter or shRNA encoding element being interrupted by DNA sequences flanked by a first and second recombinase-sensitive element in an appropriate orientation such that all or a portion of the DNA sequences is excisable upon exposure to Cre.

16. The construct of claim 15, wherein the DNA sequences flanked by a first and second recombinase-sensitive element are within the promoter.

17. The construct of claim 16, wherein the promoter is a Pol III promoter.

18. The construct of claim 17, wherein the promoter is U6 comprising a distal sequence element (DSE), proximal sequence element (PSE) and TATA box.

19. The construct of claim 18, wherein the DNA sequences flanked by a first and second recombinase-sensitive element are between the DSE and PSE.

20. The construct of claim 15, wherein the DNA sequences flanked by a first and second recombinase-sensitive element are within the shRNA encoding element.

21. The construct of claim 20, wherein the DNA sequences flanked by a first and second recombinase-sensitive element comprise a transcription termination signal.

22. The construct of any one of claims 16-21, wherein the construct further includes a spacer nucleotide sequence between the first recombinase-sensitive element and the second recombinase-sensitive element.

23. The construct of claim 22, wherein the spacer is between about 50 and 200 nucleotides in length.

24. An inducible silencing construct for the expression of a shRNA, the construct comprising a ubiquitin C promoter (UbC) operably linked to an intron comprising an shRNA encoding element, the Ubc promoter comprising a 5′ promoter region and exon 1, operably linked, wherein the construct further comprises one or more tetracycline responsive elements (TRE) within the 5′ promoter region or exon 1.

25. The construct of claim 24, further comprising a tetracycline transcriptional repressor (tTs) encoding nucleic acid sequence and an internal ribosomal entry site (IRES).

26. The construct of claim 24 or 25, further comprising a marker protein encoding nucleic acid sequence.

27. An inducible silencing construct for the expression of a shRNA, the construct comprising a ubiquitin C promoter (UbCP) operably linked to an intron, the Ubc promoter comprising a 5′ promoter region and exon 1, operably linked, wherein the intron comprises an shRNA encoding element downstream of a transcription termination signal, and wherein the construct further comprises a first loxP site in said exon 1, and a second loxP site between the transcription termination signal and the shRNA encoding element, the loxP sites being in the same orientation such that a portion of the intron comprising the transcription termination signal is excisable upon exposure to Cre.

28. The construct of claim 27 further comprising a marker protein encoding nucleic acid sequence upstream of the transcription termination site.

29. The construct of claims 27 or 28, further comprising a marker protein nucleic acid sequence downstream of the shRNA encoding element.

30. An inducible desilencing construct for the expression of a shRNA, the construct comprising a ubiquitin C promoter (UbCP) operably linked to an intron, the Ubc promoter comprising a 5′ promoter region and exon 1, operably linked, wherein the intron comprises an shRNA encoding element upstream of a transcription termination signal, and wherein the construct further comprises a first loxP site in said exon 1, and a second loxP site downstream of the transcription termination signal, the loxP sites being in the same orientation such that a portion of the intron comprising the shRNA encoding element and the transcription termination signal is excisable upon exposure to Cre.

31. The construct of claim 30, further comprising a marker protein encoding nucleic acid sequence between the shRNA encoding element and the transcription termination signal.

32. The construct of claims 30 or 31, further comprising a marker protein encoding nucleic acid sequence downstream of the second loxP site.

33. The construct of any one of claims 26, 28, 29, 31 and 32, wherein the marker protein is red or green fluorescent protein.

34. The construct of any one of claims 6-33, wherein the recombination-sensitive element is a loxP site.

35. The construct of any one of claims 1-34, wherein the shRNA comprises a sequence sufficiently complementary to a target mRNA to mediate degradation of said target.

36. The construct of claim 35, wherein said target mRNA encodes a mutant protein.

37. The construct of claim 36, wherein said mutant protein is a disease-causing mutant.

38. The construct of claim 37, wherein the mutant protein is SOD1.

39. The construct of claim 38, wherein said mutant protein is SOD1^{G93A}.

40. The construct of claim 38, wherein said mutant protein is SOD1^{G85R}.

41. The construct of any one of the preceding claims for the treatment of a disease.

42. The construct of claim 41, wherein said disease is caused by aberrant gene function.

43. The construct of claim 41, wherein said disease is a dominant, gain-of-function mutation.

44. The construct of claim 42, wherein said disease is a neurological disease.

45. A vector comprising the construct of any one of the preceding claims.

46. The vector of claim 45, wherein said vector is a viral vector.

47. The vector of claim 46, wherein said vector is an AAV or lentivirus.

48. A cell comprising a construct of any one of claims 1-44.

49. A cell comprising the vector of any one of claims 46-47.

50. The cell comprising the construct of any one of claims 1-44 and 46-47, wherein the cell is an animal cell.

51. A nonhuman transgenic animal carrying a transgene comprising the constructs of any one of claims 1-44.

52. A nonhuman homologous recombinant animal which contains cells from any one of claims 48-49.

53. A method for promoting inducible RNAi, the method comprising introducing into a cell the construct of any one of claims 1-44 under conditions such that shRNA expression is inducible.

54. The method of claim 53, wherein the cell is present in a subject.

55. The method of claim 53, wherein the cell is a cultured cell.

56. The method of claim 53, wherein said introducing comprises transfecting said cell.

57. The method of claim 53, wherein said introducing comprises infecting said cell with a viral vector.

58. A method of promoting inducible RNAi in a subject, the method comprising administering the construct of any one of claims 1-44.

59. A method for selectively inhibiting mutant gene expression in vivo or in vitro, the method comprising introducing into a host cell the construct of any one of claims 1-44 under conditions such that said shRNA is expressed, thereby inhibiting mutant gene expression.

60. The method of claim 59, wherein the shRNA does not inhibit expression of the wild type allele.

61. A method for treating a disease in a subject, the method comprising administering the construct of any one of claims 1-44, thereby treating a disease in a subject.

62. The method of claim 61, wherein the disease is caused by aberrant gene function.

63. The method of claim 61, wherein the disease is caused by a mutation that is a dominant, gain-of-function mutation.

64. A method for identifying a compound which modulates RNAi, the method comprising:
(a) contacting a cell comprising the construct of any one of claims 1-44 with a test compound; and
(b) determining the effect of the test compound on an indicator of RNAi activity in said cell, thereby identifying a compound which modulates RNAi.

65. A compound identified according to the method of claim 64.

66. A method for modulating RNAi, the method comprising contacting a cell expressing the construct of any one of claims 1-44 with the compound of claim 65 in a sufficient concentration to modulate the activity of RNAi.

67. A method for modulating RNAi, the method comprising contacting a cell expressing the construct of any one of claims 48-49 with a compound which binds to said construct in a sufficient concentration to modulate the activity of RNAi.

68. A method for deriving information about the function of a gene in a cell or organism comprising:
(a) introducing into said cell or organism the construct of any one of claims 1-44;
(b) maintaining the cell or organism under conditions such that RNAi can occur;
(c) determining a characteristic or property of said cell or organism; and
(d) comparing said characteristic or property to a suitable control, the comparison yielding information about the function of the gene.

69. A method of validating a candidate protein as a suitable target for drug discovery comprising:
(a) introducing into a cell or organism the construct of any one of claims 1-44;
(b) maintaining the cell or organism under conditions such that RNAi can occur;
(c) determining a characteristic or property of said cell or organism; and
(d) comparing said characteristic or property to a suitable control,
the comparison yielding information about whether the candidate protein is a suitable target for drug discovery.

70. A kit comprising reagents for activating RNAi in a cell or organism, said kit comprising:
(a) the construct of any one of claims 1-44; and
(b) instructions for use.

71. A method of excising a DNA sequence, the method comprising:
(a) exposing the construct of any one of claims 1-23 and 27-33 to Cre recombinase;
(b) allowing recombination; thereby excising a portion of said DNA sequence.

72. A method of promoting target gene expression, the method comprising:
(a) exposing the construct of any one of claims 1-3, 6-14 and 30-32 to a Cre recombinase;
(b) excising of a portion of the shRNA flanked by loxP sites; and
(c) disrupting expression of the shRNA, thereby allowing the target gene to be expressed.

73. The method of claim 72, wherein said disrupted expression results in the silencing of a mutant gene.

74. The method of claim 73, wherein the mutant gene is SOD1.

75. A method of recovering promoter function, the method comprising exposing the construct of any one of claims 5 and 15-19 to a Cre recombinase protein under conditions such that shRNA expression is activated, thereby recovering said promoter function.

76. A method of disrupting promoter function, the method comprising
(a) exposing the construct of any one of claims 1, 3 and 12-14 to a Cre recombinase;
(b) allowing recombination, thereby disrupting promoter function.

77. A method of inhibiting expression of a target gene, the method comprising:
(a) exposing the construct of any one of claims 5 and 15-19 to a Cre recombinase;
(b) activating said promoter; and
(c) expressing said shRNA, thereby inhibiting target gene expression.

78. The method of claim 76 or 77, wherein said promoter may be regulated in an animal.

79. The method of claim 78, wherein said promoter is regulated temporally.

80. The method of claim 78, wherein said promoter is regulated spatially.


SEQUENCE LISTING (9 sequences)

SEQ ID NO: 1 - DNA, 106 bp, Artificial Sequence
  synthetic shRNA
tgctgttgacagtgagcgaccaagggagattcgttgcaactcagtgaagccacagatgtgagttgtaacatctcccttggctgcctactgcctcggacttcaaggg

SEQ ID NO: 2 - DNA, 3369 bp, Artificial Sequence
  expression construct
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgctagcaatctcgagccttggctagaactagtggatccgacgccgccatctctaggcccgcgccggccccctcgcacagacttgtgggagaagctcggctactcccctgccccggttaatttgcatataatatttcctagtaactatagaggcttaatgtgcgataaaagacagataatcagatctataacttcgtatagcatacattatacgaagttatagatctctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgacaaagatgctgtggccgataagcttatcggccacagcatctttgtctttttgaattcaataacttcgtatagcatacattatacgaagttatgcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 3 - DNA, 4388 bp, Artificial Sequence
  expression construct
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgctagcaatctcgagccttggctagaactagtggatccgacgccgccatctctaggcccgcgccggccccctcgcacagacttgtgggagaagctcggctactcccctgccccggttaatttgcatataatatttcctagtaactatagaggcttaatgtgcgataaaagacagataatcagatctaataacttcgtatagcatacattatacgaagttatattaagggttccggatctcgaggcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatgctagcaggtcgagggacctaataacttcgtatagcatacattatacgaagttatagatctctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgacaaagatgctgtggccgataagcttatcggccacagcatctttgtctttttgaattcatgcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 4 - DNA, 2299 bp, Artificial Sequence
  expression construct
ttaaacagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggaccgaagcgtgtggagagaccgccangggctgtagtctgggtcccgcgagcaaggttgccctgaactgaggggttgggggggagcgcagcaaaatggcggctgttcccgagtctgaatggaagacgcctgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggtaccgaattcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgatatcatctgcaggcggccgcagaacaaaaactcatctcagaagaggatctggtgcagccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagaccggttagcaggcatgctggggatgcggt

SEQ ID NO: 5 - DNA, 2301 bp, Artificial Sequence
  expression construct
ttaaacagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgctcgatccctatcagtgatagagatccctatcagtgatagagactagcgcacagctagttccgtcgcagccgggatttgggtcgcggttcttgtttgtggatcgctgtgatcgtcacttggtgagtagcgggctgctgggctggccggggctttcgtggccgccgggccgctcggtgggaccgaagcgtgtggagagaccgccangggctgtagtctgggtcccgcgagcaaggttgccctgaactgaggggttgggggggagcgcagcaaaatggcggctgttcccgagtctgaatggaagacgcctgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggtaccgaattcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttctttagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgatatcatctgcaggcggccgcagaacaaaaactcatctcagaagaggatctggtgcagccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtat

SEQ ID NO: 6 - DNA, 2300 bp, Artificial Sequence
  expression construct
ttaaacagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtctcgatccctatcagtgatagagatccctatcagtgatagagactagcttgtggatcgctgtgatcgtcacttggtgagtagcgggctgctgggctggccggggctttcgtggccgccgggccgctcggtgggaccgaagcgtgtggagagaccgccangggctgtagtctgggtcccgcgagcaaggttgccctgaactgaggggttgggggggagcgcagcaaaatggcggctgttcccgagtctgaatggaagacgcctgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggtaccgaattcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttctttagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgatatcatctgcaggcggccgcagaacaaaaactcatctcagaagaggatctggtgcagccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtat

SEQ ID NO: 7 - DNA, 3850 bp, Artificial Sequence
  expression construct
ttaaacagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtctcgatccctatcagtgatagagatccctatcagtgatagagactagcttgtggatcgctgtgatcgtcacttggtgagtagcgggctgctgggctggccggggctttcgtggccgccgggccgctcggtgggaccgaagcgtgtggagagaccgccangggctgtagtctgggtcccgcgagcaaggttgccctgaactgaggggttgggggggagcgcagcaaaatggcggctgttcccgagtctgaatggaagacgcctgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggtacctgctgttgacagtgagcgaccaagggagattcgttgcaactcagtgaagccacagatgtgagttgtaacatctcccttggctgcctactgcctcggacttcaagggaattcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttctttagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgatatcaccatgtccagattagataaaagtaaagtgattaacagcgcattagagctgcttaatgaggtcggaatcgaaggtttaacaacccgtaaactcgcccagaagctaggtgtagagcagcctacattgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagcattaatatttggattttctgcacagttagatgaaaaaaagcatacacccattgaggatggtaataaaccaaaaaagaagagaaagctggcagtgtcagtgacatttgaagatgtggctgtgctctttactcgggacgagtggaagaagctggatctgtctcagagaagcctgtaccgtgaggtgatgctggagaattacagcaacctggcctccatggcaggattcctgtttaccaaaccaaaggtgatctccctgttgcagcaaggagaggatccctggtaagcggcctcgagctcaagcttcgaattctgcagtcgacggtaccgcgggcccgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttgccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagaccggttagcaggcatgctggggatg

SEQ ID NO: 8 - DNA, 4300 bp, Artificial Sequence
  expression construct
ttaaacagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtctcgaaataacttcgtatagcatacattatacgaagttatactcgaggctagcttgtggatcgctgtgatcgtcacttggtgagtagcgggctgctgggctggccggggctttcgtggccgccgggccgctcggtgggaccgaagcgtgtggagagaccgccangggctgtagtctgggtcccgcgagcaaggttgccctgaactgaggggttgggggggagcgcagcaaaatggcggctgttcccgagtctgaatggaagacgcctgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggtaccgaattcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttctttagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgataccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagaccggaaataacttcgtatagcatacattatacgaagttatctagcttgtggatcgctgtgatcgtcacttggtgagtagcgggctgctgggctggccggggctttcgtggccgccgggccgctcggtgggaccgaagcgtgtggagagaccgccangggctgtagtctgggtcccgcgagcaaggttgccctgaactgaggggttgggggggagcgcagcaaaatggcggctgttcccgagtctgaatggaagacgcctgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggtacctgctgttgacagtgagcgaccaagggagattcgttgcaactcagtgaagccacagatgtgagttgtaacatctcccttggctgcctactgcctcggacttcaagggaattcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttctttagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgatatcatctgcaggcggccgcagaacaaaaactcatctcagaagaggatctggtgcagccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagaccggttagcaggcatgctggggatgcggtgggctctatggcttctgaggc

SEQ ID NO: 9 - DNA, 4200 bp, Artificial Sequence
  expression construct
ttaaacagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtctcgaaataacttcgtatagcatacattatacgaagttatctagcttgtggatcgctgtgatcgtcacttggtgagtagcgggctgctgggctggccggggctttcgtggccgccgggccgctcggtgggaccgaagcgtgtggagagaccgccangggctgtagtctgggtcccgcgagcaaggttgccctgaactgaggggttgggggggagcgcagcaaaatggcggctgttcccgagtctgaatggaagacgcctgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggtacctgctgttgacagtgagcgaccaagggagattcgttgcaactcagtgaagccacagatgtgagttgtaacatctcccttggctgcctactgcctcggacttcaagggaattcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttctttagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgatatcatctgcaggcggccgcagaacaaaaactcatctcagaagaggatctggtgcagccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaaccacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagaccggtaataacttcgtatagcatacattatacgaagttatgctagcttgtggatcgctgtgatcgtcacttggtgagtagcgggctgctgggctggccggggctttcgtggccgccgggccgctcggtgggaccgaagcgtgtggagagaccgccangggctgtagtctgggtcccgcgagcaaggttgccctgaactgaggggttgggggggagcgcagcaaaatggcggctgttcccgagtctgaatggaagacgcctgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggtaccgaattcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttctttagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgatgtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagaccggactcgag